(19) United States
(12) Patent Application Publication
(10) Pub. No.: US 2010/0227778 A1
(43) Pub. Date: Sep. 9, 2010

(54) Diagnosis and Treatment of Noonan Syndrome and Neoplastic Disorders

(75) Inventors: Benjamin G. Neel, Toronto (CA); Amy E. Roberts, Charlestown, MA (US); Raju Kucherlapati, Wayland, MA (US); Kenneth D. Swanson, Brookline, MA (US); Toshiyuki Araki, Toronto (CA)

Correspondence Address: FISH & RICHARDSON PC, P.O. BOX 1022, MINNEAPOLIS, MN, 55440-1022, US

(73) Assignees: The Brigham and Women's Hospital, Inc., Boston, MA (US); Beth Israel Deaconess Medical Center, Inc., Boston, MA (US)

(21) Appl. No.: 12/377,909

(22) PCT Filed: Aug. 17, 2007
(86) PCT No.: PCT/US07/76248

§ 371 Date: May 25, 2010

Related U.S. Application Data
(60) Provisional application No. 60/838,662, filed on Aug. 18, 2006.
(60) Provisional application No. 60/845,564, filed on Sep. 19, 2006.

(51) Int. Cl. C40B 50/00; G01N 33/53; C12Q 1/68

(52) U.S. Cl. 506/23; 435/7.1; 435/6; 435/7.2

(57) ABSTRACT

Methods and compositions for diagnosing and treating Noonan syndrome and neoplastic disorders are provided herein.

### CROSS-REFERENCE TO RELATED APPLICATIONS

This application claims the benefit of priority of U.S. Ser. No. 60/838,662, filed Aug. 18, 2006, and U.S. Ser. No. 60/845,564, filed Sep. 19, 2006, The contents of the prior applications are hereby incorporated by reference in their entirety.

### FEDERALLY SPONSORED RESEARCH OR DEVELOPMENT

The work described herein was funded, in part through grants from the National Institutes of Health (grants R37CA49152, DE16140, and MO1-RR02172). The United States government may, therefore, have certain rights in the invention.

### TECHNICAL FIELD

This invention relates to methods and compositions for diagnosis and treatment of genetic disorders, and more particularly to diagnosis and treatment of Noonan syndrome, and neoplastic disorders.

### BACKGROUND

Noonan syndrome (NS) is the most common single-gene cause of congenital heart disease (CHD), and also frequently includes short stature, characteristic facial features, learning problems, and an increased risk of certain leukemias (Tartaglia and Gelb, Annu Rev Genomics Hum Genet, 6:45-68, 2005). Consistent with its autosomal dominant inheritance pattern, gain-of-function mutations in the PTPN11 gene, encoding the tyrosine phosphatase SHP2, cause ˜50% of NS cases. SHP2 is required for full activation of the RAS/ERK MAP kinase (MAPK) cascade downstream of most growth factor and cytokine receptors, and NS mutants enhance ERK activation ex vivo and in mice (Fragale et al., Hum. Mut., 23: 267-277, 2004; Kontaridis et al., J. Biol. Chem., 281:6785-6792, 2005; Araki et al., Nat. Med., 10:849-857, 2004). KRAS mutations account for <5% of NS (Schubbert et al., Nat. Genet., 18:331-336, 2006), but the gene(s) responsible for the remainder of NS cases remain unknown.

### SUMMARY

The invention is based, in part, on the discovery of mutations within the human Son of sevenless 1 (SOS1) gene which are associated with human disease. The invention provides, inter alia, methods and compositions for diagnosing and treating human disorders including NS and neoplastic disorders.

In one aspect, the invention features a method for diagnosing in a subject, or identifying a subject at risk for, Noonan syndrome (NS). The method includes, for example, determining if one or more mutations are present in a SOS gene (e.g., SOS1) of the subject, wherein the presence of one or more mutations indicates that the subject is affected with, or at risk for, NS. In one embodiment, the subject is a subject who presents with one or more phenotypic characteristics of NS. Phenotypic characteristics of NS include dysmorphic facial features (e.g., broad forehead, hypertelorism, down-slanting palpebral fissures, highly arched palate, low set and posteriorly rotated cars), proportionate short stature, pedals deformity, cryptorchidism, developmental delay, genitourinary malformations, bleeding disorders, lymphatic dysplasia, growth failure, and cardiac defects (e.g., hypertrophic cardiomyopathy, pulmonic stenosis, atrial septal defect, and aortic coarctation).

In one embodiment, the subject has been screened for a mutation in the PTPN11 gene (e.g., the subject has been identified as lacking a mutation in the PTPN11 gene, prior to screening for SOS1 mutations).

The method can further include determining whether a PTPN11 and/or a KRAS gene of the subject has a mutation. For example, the method includes screening for mutations in SOS1 genes, PTPN11, and KRAS genes of the subject.

In various embodiments of the methods, subjects are evaluated for mutations which are substitutions, deletions, or insertions of one or more nucleotides in a SOS1 gene. In one embodiment, the mutation includes a mutation of a single nucleotide, e.g., a substitution, deletion, or insertion of a single nucleotide. In one embodiment, the mutation includes a missense mutation. Subjects may be evaluated for mutations which are chromosomal rearrangements (e.g., translocations or deletions, such as translocations or deletions which activate SOS1 by juxtaposing a regulatory sequence with a SOS1 coding sequence),

Exemplary mutations indicative of NS (or risk for NS) include mutations at one of the following nucleotide positions of the SOS1 sequence of SEQ ID NO:1: 797, 806, 925, 1010, 1358, 1642, 1654, 1964, and 2536. In some embodiments, the mutation is other than one of the following mutations: an insertion between nucleotides 3248 and 3249, or a mutation at nucleotide 3032.

In one embodiment, the mutation indicative of NS is a substitution in the SOS1 coding sequence, e.g., corresponding to one of the following substitutions in SEQ ID NO:1: 797C>A, 806T>G, 925G>T, 1010A>G, 1358G>C, 1642A>C, 1654A>G, 1964C>T, or 2536G>A.

In one embodiment, the mutation in the SOS1 gene results in the substitution, deletion, or insertion of one or more amino acids of the polypeptide encoded by the gene. For example, the mutation results in a mutation at one or more of the following amino acid positions in the SOS1 polypeptide of SEQ ID NO:2: T266, M269, D309, Y337, G434, S548, R552, P655, or E846. The substitution can include one of the following substitutions: T266K, M269R, D309Y, Y337C, G434R, S548R, R552G, P655L, or E846K,

In one embodiment, the mutation in the SOS1 gene results in a mutation in one of the following domains of the polypeptide encoded by the gene: the Dbl Homology (DH) domain, the Pleckstrin Homology (PH) domain, the Helical Linker (HL) domain, the Ras Exchange Motif (REM) domain, or the Cdc25 domain.

In one embodiment, the mutation in the SOS1 gene results in an increased level of expression or activity of the polypeptide encoded by the gene. For example, the polypeptide encoded by the gene mediates enhanced Ras, Erk, and/or Rac activation, relative to a control (e.g., relative to a wild type SOS1 polypeptide),

In one embodiment, the mutation includes a mutation in an exon of the SOS1 gene (e.g., a mutation in exon 6, exon 7, exon 8, exon 9, exon 10, exon 12, exon 16, or exon 19).

In another embodiment, the mutation is a mutation in a promoter, enhancer, untranslated region (UTR), or intron of the gene (e.g., a 3′UTR, 5′UTR).

The method of determining whether a subject has a mutation in a SOS1 gene can include determining the identity of at least one nucleotide in the SOS1 gene of the subject (e.g., wherein the nucleotide in the SOS1 gene is in an exon, intron, regulatory. 3′UTR, and/or 5′UTR region of the gene, e.g., wherein a the identify of at least 50, 100, 250, 500, 1000, 2500, or 5000 nucleotides of the SOS1 gene is determined). Alternatively, or in addition, the method can include determining whether the subject contains as marker (e.g., a polymorphism) which is linked to a SOS1 mutation.

In one embodiment, the sequence of one or more exons, or portions thereof, of a SOS1 gene of the subject is determined. For example, the sequence of one or more of the following exons is determined: exon 6, exon 7, exon 8, exon 9, exon 10. exon 12, exon 16, or exon 19.

In one embodiment, the detecting includes detecting increased expression or activity of a SOS1 polypeptide encoded by a SOS1 gene of the subject. Optionally, the method further includes determining a sequence in the SOS1 gene of the subject.

The method can further include determining whether the subject presents with one or more phenotypic characteristics of Noonan syndrome (e.g., wherein the diagnosis of Noonan syndrome is made in conjunction with an evaluation of presentation of one or more of the characteristic features).

The method can be used to distinguish Noonan syndrome from a related disorder, such as Cardiofaciocutaneous syndrome, or Costello syndrome.

The subject is for example, a fetal, neonatal, juvenile, or adult subject.

In various embodiments, SOS2, or a gene encoding a product in a SOS signaling pathway (e.g., RAS, RAF, MEK, ERK) is examined in the method (e.g., instead of, or in addition to SOS1). Other SOS genes and homologs thereof can be examined.

In another aspect, the invention features a method for diagnosing in a subject, or identifying a subject at risk for, NS. The method includes, for example, determining if one or more mutations are present in a SOS1 polypeptide of the subject, wherein the presence of one or more mutations indicates that the subject is affected with, or at risk for, NS.

The foregoing methods can further include making a decision about further evaluation (e.g., further diagnostic testing) of the subject based on the determining (e.g., based on whether or not the subject has a mutation in a SOS1 gene). In one embodiment, a mutation in a SOS1 gene or polypeptide is not detected and a decision is made not to further evaluate the subject for symptoms of NS. In one embodiment, one or more mutations are detected and a decision is made to further evaluate the subject for symptoms of NS. The further evaluation can include one or more of cardiovascular evaluation, testing by echocardiogram or EKG, testing for a bleeding disorder, testing for renal anomalies, hearing examination, eye examination, and cognitive evaluation.

The detecting includes detecting increased expression or activity of a SOS1 polypeptide encoded by a SOS1 gene of the subject, and/or detecting a change in the SOS1 polypeptide (e.g., a change a biochemical characteristic of the SOS1 polypeptide, such as a change in the molecular size, antibody-binding, or Ras-binding characteristics of the SOS1 polypeptide), relative to a control, e.g., relative to a wild type SOS1 polypeptide. The method can further include determining the identity of at least one nucleotide in the SOS1 gene of the subject (e.g., wherein the nucleotide in the SOS1 gene is in an exon, intron, regulatory, 3′UTR, and/or 5′UTR region of the gene, e.g., wherein a the identify of at least 50, 100, 250, 500, 1000, 2500, or 5000 nucleotides of the SOS1 gene is determined).

In another aspect, the invention features a method of evaluating in a subject risk of developing or suffering from pulmonary stenosis, wherein the subject is a subject at risk for NS. The method includes determining whether one or more mutations are present in a SOS1 gene of the subject, wherein the presence of one or more mutations indicates that the subject is at risk for developing, or is affected by, pulmonary stenosis. The method can further include determining whether one or more mutations are present in a second gene (e.g., a PTPN11 gene) of the subject. The method can further include evaluating the subject for symptoms of pulmonary stenosis or atrial septal defect (e.g., and administering a treatment to the subject, based on the evaluating).

The invention also features a method of evaluating in a subject risk of developing or suffering from atrial septal defect, wherein the subject is a subject at risk for NS. The method includes determining whether one or more mutations are present in a SOS1 gene of the subject, wherein the presence of one or more mutations indicates that the subject is less likely to develop, or be affected by, an atrial septal defect. The method can further include determining whether one or more mutations are present in a second gene (e.g., a PTPN11 gene) of the subject. The method can further include evaluating the subject for symptoms of pulmonary stenosis or atrial septal defect (e.g., and administering a treatment to the subject, based on the evaluating).

In another aspect, the invention features a method of diagnosing NS in a subject (or distinguishing NS from related syndromes). The method includes providing a subject having one or more characteristics or symptoms of NS, cardio-facial-cutaneous syndrome, or Costello syndrome, and determining whether one or more mutations are present in a SOS1 gene of the subject, wherein the presence of a mutation indicates that the subject has, or is more likely to have, NS as opposed to a related syndrome such as cardio-facial-cutaneous syndrome, or Costello syndrome.

If the subject has a mutation in a SOS1 gene, the method can further include evaluating the subject for further characteristics of NS and/or further evaluation for a symptom of a related syndrome such as cardio-facial-cutaneous syndrome, or Costello syndrome.

The method can further include determining whether the subject has a mutation in a second gene (e.g., PTPN11, KRAS, BRAF, MEK1, MEK2).

In another aspect, the invention features a method for diagnosing or evaluating in a subject, or identifying a subject at risk for, a neoplastic disorder. The method includes, for example, determining whether one or more mutations are present in a SOS gene (e.g., SOS1, SOS2) and/or a SOS polypeptide of the subject, wherein the presence of a mutation indicates that the subject is affected by, or at risk for, a neoplastic disorder. Alternatively, or in addition, the method includes determining whether one or more mutations are present in a gene encoding a product in a SOS signaling pathway (e.g., Ras, Rac, Erk, are related polypeptides).

In various embodiments, the neoplastic disorder is a breast cancer, a neoplastic disorder of hematopoietic cells (e.g., a neoplastic disorder of hematopoietic cells selected from the following: T-Cell Acute Lymphoblastic Leukemia (T-ALL), acute myelogenous leukemia (AML), juvenile myelomonocytic leukemia (JMML) (e.g., JMML which is not associated with a PTPN11 mutation), and Myelodysplastic and Myeloproliferative Syndrome (MDS/MPS)), a neoplastic disorder of the brain or neuronal tissue (e.g., neuroblastoma or glioblastoma/astrocytoma), a carcinoma (e.g., an adenocarcinomas; a carcinoma of breast, lung, or colon tissue), a bladder cancer, or a skin cancer (e.g., a melanoma).

In one embodiment, the method further includes determining whether a second cancer-associated gene of the subject includes a mutation (e.g., wherein the method includes screening for mutations in other genes of the subject).

In various embodiments of the methods, subjects are evaluated for mutations which are substitutions, deletions, or insertions of one or more nucleotides in a SOS1 gene. In one embodiment, the mutation includes a mutation of a single nucleotide, e.g., a substitution, deletion, or insertion of a single nucleotide. In one embodiment, the mutation includes a missense mutation. Subjects may be evaluated for mutations which are chromosomal rearrangements (e.g., translocations or deletions, such as translocations or deletions which activate SOS1 by juxtaposing a regulatory sequence with a SOS1 coding sequence).

Exemplary mutations indicative of a neoplastic disorder (or risk far a neoplastic disorder) include mutations at one of the following nucleotide positions of the SOS1 sequence of SEQ. ID NO:1: 797, 806, 925, 1010, 1358, 1642, 1654, 1964, and 2536. In some embodiments, the mutation is other than one of the following mutations: an insertion between nucleotides 3248 and 3249, or a mutation at nucleotide 3032.

In one embodiment, the mutation indicative of a neoplastic disorder is a substitution in the SOS1 coding sequence, e.g., corresponding to one of the following substitutions in SEQ ID NO:1: 797C>A, 806T>G, 925G>T, 1010A>G, 1358G>C, 1642A>C, 1654A>G, 1964C>T, or 2536G>A,

In one embodiment, the mutation in the SOS1 gene results in the substitution, deletion, or insertion of one or more amino acids of the polypeptide encoded by the gene. For example, the mutation results in a mutation at one or more of the following amino acid positions in the SOS1 polypeptide of SEQ ID NO:2: T266, M269, D309, Y337, G434, S548, R552, P655, or E846. The substitution can include one of the following substitutions: T266K, M269R, D309Y, Y337C, G434R, S548R, R552G, P655L, or E846K.

In various embodiments, the mutation indicative of a neoplastic disorder includes a mutation at one of the following nucleotide positions of the SOS1 sequence of SEQ ID NO:1: 947, 1018, 1429, 1964, 2050, 2416, 2581, or 3056. The mutation can include a substitution, such as one of the following substitutions: 947C>T, 1018C≧C, 1429G>T, 1964C>T, 2050C>T, 2416G>A, 2581G>A, or 3056G>A.

In various embodiments, the mutation results in a mutation at one or more of the following amino acid positions in the SOS1 polypeptide of SEQ ID NO:2: S316, P340, Q477, P655, P684, G806, V861, or R1019. The substitution can include one of the following substitutions: S316L, P340S, Q477 changed to a stop codon, P655L, P684S, G806R, V861I, or R1019Q.

In various embodiments, the mutation indicative of a neoplastic disorder includes a mutation described in Table D, below.

In various embodiments, the mutation results in a truncated SOS1 polypeptide a SOS1 polypeptide which includes the histone fold, the DH domain, and lacks at least one other domain).

In one embodiment, the mutation in the SOS1 gene results in a mutation in one of the following domains of the polypeptide encoded by the gene: the Dbl Homology (DH) domain, the Pleckstrin Homology (PH) domain, the Helical Linker (HL) domain, the Ras Exchange Motif (REM) domain, or the Cdc25 domain.

In one embodiment, the mutation in the SOS1 gene results in an increased level of expression or activity of the polypeptide encoded by the gene. For example, the polypeptide encoded by the gene mediates enhanced Ras, Erk, and/or Rac activation, relative to a control (e.g., relative to a wild type SOS1 polypeptide).

In one embodiment, the mutation includes a mutation in an exon of the SOS1 gene (e.g., a mutation in exon 6, exon 7, exon 8, exon 9, exon 10, exon 12, exon 16, or exon 19).

In another embodiment, the mutation is a mutation in a promoter, enhancer, untranslated region (UTR), or intron of the gene (e.g., as 3′UTR, 5′UTR).

The method of determining whether a subject has a mutation in a SOS1 gene can include determining the identity of at least one nucleotide in the SOS1 gene of the subject (e.g., wherein the nucleotide in the SOS1 gene is in an exon, intron, regulatory, 3′UTR, and/or 5′UTR region of the gene, e.g., wherein a the identify of at least 50, 100, 250, 500, 1000, 2500, or 5000 nucleotides of the SOS1 gene is determined). Alternatively, or in addition, the method can include determining whether the subject contains a marker (e.g., a polymorphism) which is linked to a SOS1 mutation.

In one embodiment, the sequence of one or more exons, or portions thereof, of a SOS1 gene of the subject is determined. For example, the sequence of one or more of the following exons is determined: exon 6, exon 7, exon 8, exon 9, exon 1.0, exon 12, exon 16, or exon 19.

The method can include detecting increased expression or activity of a SOS1 polypeptide encoded by a SOS1 gene of the subject.

The method can further include determining a sequence in the SOS1 gene of the subject.

In various embodiments, the method further includes determining whether the subject presents with one or more symptoms of to neoplastic disorder (e.g., wherein the diagnosis of the neoplastic disorder is made in conjunction with an evaluation of presentation of one or more of symptoms of the neoplastic disorder).

In another aspect, the invention features a method for diagnosing in a subject, or identifying a subject at risk for, Noonan syndrome. The method includes evaluating the expression or activity of a SOS1 polypeptide in a sample from the subject, relative to a control, wherein an increase in the expression or activity of the SOS1 polypeptide relative to the control is indicative of Noonan syndrome. For example, enhanced Ras and/or Erk activation mediated by the SOS1 polypeptide, relative to a control, is indicative of Noonan syndrome (e.g., relative to as wild type SOS1 polypeptide).

In another aspect, the invention features a method for identifying an agent that modulates the activity of a SOS1 polypeptide. The method includes providing a sample which includes a SOS1 (e.g., a mutant SOS1 polypeptide which has increased activity relative to a control), contacting the sample with a test compound under conditions in which the SOS1 polypeptide is active, and evaluating the activity of the SOS1 polypeptide in the presence of the test compound, wherein a change in the activity of the SOS1 polypeptide indicates that the test compound is an agent that modulates the activity of the SOS1 polypeptide.

The method can further include: evaluating the compound for an effect on cell growth (and/or another characteristic of neoplastic transformation, such as cell survival, tumorigenic/metastatic potential, resistance to apoptosis, and anchorage-independent growth); and/or evaluating the compound in an animal model for a neoplastic disorder; and/or evaluating an effect of the compound on a symptom of Noonan syndrome,

In another aspect, the invention features a method for genotyping a subject. The method includes determining the identity of at least one nucleotide of a SOS gene or SOS pathway gene (e.g., SOS1, SOS2) of a subject, and creating a record which includes information about the identity of the nucleotide and information relating to a genotypic or phenotypic characteristic of Noonan syndrome or a neoplastic disorder in the subject.

In one embodiment, the method further includes comparing the information in the record to reference information (e.g., information about a corresponding nucleotide from a reference sequence).

In one embodiment, the method further includes comparing the nucleotide to a corresponding nucleotide from a genetic relative or family member (e.g., a parent, grandparent, sibling, progeny, prospective spouse, etc.).

In one embodiment, the method further includes evaluating risk or determining diagnosis of Noonan syndrome or a neoplastic disorder in the subject as a function of the information in the record. The method can further include recording information about the identity of the nucleotide and the genotypic or phenotypic characteristic of Noonan syndrome or the neoplastic disorder, e.g., in a database.

In one embodiment, the identity of a plurality of nucleotides of the SOS gene are determined (ex., at least 10, 20, 50, 100, 500, or 1000 nucleotides are evaluated (e.g., consecutive or non-consecutive)).

The method can further include making a decision about whether to provide a treatment as a function of information in the record.

In another aspect, the invention features a method for treating or preventing Noonan syndrome in a subject. The method includes identifying a subject diagnosed with or at risk for Noonan syndrome; and administering to the subject an agent that modulates the activity of a SOS1 polypeptide, or a polypeptide in a SOS signaling pathway (e.g., Ras, Raf, MEK, Erk, Rsk, PI3-Kinase, Akt, Tor, Rac).

For example, the agent is administered in an amount effective to reduce SOS1 activity (or activity of a polypeptide in a SOS signaling pathway) in a cell of the subject.

In one embodiment, the agent is administered in an amount effective to reduce or ameliorate at least one symptom of Noonan syndrome.

The identifying can include evaluating a genotypic or phenotypic characteristic of Noonan syndrome in the subject (e.g., a genetic, biochemical, anatomical, or cognitive feature or a symptom of Noonan syndrome). For example, the feature of Noonan syndrome is a genetic mutation associated with Noonan syndrome, e.g., a mutation to a SOS1 gene.

In another aspect, the invention features a method for treating or preventing a neoplastic disorder in a subject. The method includes identifying a subject diagnosed with or at risk for a neoplastic disorder; and administering to the subject an agent that modulates SOS1 activity, or activity of a polypeptide in a SOS signaling pathway (e.g., Ras, Raf, MEK, Erk, Rsk, PI3-Kinase, Akt, Tor, Rac).

For example, the agent is administered in an amount effective to reduce. SOS1 activity (or activity of a polypeptide in a SOS signaling pathway) in a cell of the subject.

In one embodiment, the agent is administered in an amount effective to reduce or ameliorate at least one symptom of the neoplastic disorder.

The identifying can include evaluating a genotypic or phenotypic characteristic of the neoplastic disorder in the subject (e.g., a genetic, biochemical, or symptom of the disorder). For example, the characteristic of the neoplastic disorder is a genetic mutation associated with the neoplastic disorder, e.g., in a SOS1 gene.

In another aspect, the invention features a kit for diagnosing in a subject, or identifying a subject at risk for, Noonan syndrome. The kit includes: a nucleic acid that specifically hybridizes to or adjacent to a sequence having a mutation in a SOS1 gene, e.g., a mutation described herein. The kit can further include a second nucleic acid that hybridizes to or adjacent to a mutation in a second gene (e.g., PTPN11 or KRAS). The kit can include a pair of nucleic acids suitable for amplification of a selected region of a SOS1 gene (e.g., a region of a SOS1 gene which can include a mutation associated with NS).

In another aspect, the invention features an isolated nucleic acid molecule including the sequence of SEQ ID NO:1, or portion thereof, with least one nucleotide change (e.g., wherein the sequence is at least 80%, 85%, 90%, 95%, 96%, 97%, 98%, or 99% identical to SEQ ID NO:1, or portion thereof, and has at least one nucleotide change).

In one embodiment, the nucleotide change results in a mutation at one or more amino acid positions of the polypeptide encoded by the nucleic acid molecule (e.g., the nucleic acid inolecule includes a sequence encoding a mutant SOS1 polypeptide which has IC increased activity relative to a wild type SOS1 polypeptide).

The invention also features an isolated nucleic acid including a sequence encoding a mutant SOS1 polypeptide, or portion thereof, wherein the mutant SOS1 polypeptide has a mutation at one or more amino acid positions relative to a wild type SOS1 polypeptide sequence, or portion thereof (e.g., relative to the sequence of SEQ ID NO:1) (e.g., wherein the mutant SOS1 polypeptide is at least 80%, 85%, 90%, 95%, 96%, 97%, 98%, or 99% identical to SEQ ID NO:2 and has at least one amino acid change).

In one embodiment, the mutant SOS1 polypeptide has increased activity, relative to a control (e.g., activation of Erk or Ras is enhanced in the presence of the mutant SOS1 polypeptide, relative to a control).

The invention also features an isolated mutant SOS1 polypeptide, or portion thereof, including a mutation at one or more amino and positions relative to a wild type SOS1 polypeptide (e.g., relative to the sequence of SEQ ID NO:2).

In another aspect, the invention features an array which includes a substrate having a plurality of addressable areas, wherein one or more of the addressable areas includes a probe that can be used to detect a mutation in a SOS gene or SOS gene product (e.g., SOS1, SOS2) (e.g., a nucleic acid probe). The array can further include a probe that can be used to detect a mutation in a PTPN11 or KRAS gene.

In one embodiment, the array includes a plurality of probes for detecting as plurality of mutations in a SOS1 gene.

As used herein, the term “mutation” generally refers to any variation in sequence at a given position or region of nucleic acid sequence between individuals in a population, e.g., human individuals. Variations include nucleotide substitutions (e.g., transitions and transversions), insertions, deletions, inversions, and other rearrangements. A variation can encompass one or more nucleotide positions in a reference sequence that are absent, altered, inverted, or otherwise rearranged in another sequence. Some exemplary mutations cause one or more change in the amino acid sequence of an encoded protein. Other exemplary mutations can affect regulation, e.g., transcription, translation, splicing, mRNA or protein stability, protein function, mRNA or protein localization, chromatin organization, and so forth. Still other exemplary mutations are silent or are only manifest under particular circumstances. Silent mutations can be useful, e.g., as indicators. For example, they may be tightly linked to a marker that is causative of a particular property.

As used herein, “genotyping” refers to any method of evaluating genetic material. Genotyping includes a method of determining the identity of one or more nucleotides (a consecutive or non-consecutive positions), sequencing a region of nucleic acid, and determining the type and number of alleles and/or polymorphisms present in genetic material, e.g., genetic material from a subject. Exemplary methods of genotyping determined by nucleic acid sequencing, PCR or RT-PCR amplification, genotyping by one of many different technologies now commercially available such as those provided by Sequenom, Affymetrix, Illumina, Parallele, Luminex, Nimblegen and others, protein sequencing (thereby inferring nucleic acid sequence), examination of a protein, or by other methods available to those skilled in the art.

The term “biological sample” is intended to include tissues, cells and biological fluids isolated from is subject, as well as tissues, cells and fluids present within a subject.

As used herein, the term “nucleic acid molecule” includes DNA molecules (e.g., a cDNA or genomic DNA), RNA molecules (e.g., an mRNA, a dsRNA, e.g., an siRNA) and analogs of the DNA or RNA. A DNA or RNA analog can be synthesized from nucleotide analogs. The nucleic acid molecule can be single-stranded or double-stranded, e.g., double-stranded DNA or a double-stranded RNA.

The term “isolated nucleic acid molecule” or “purified nucleic acid molecule” includes nucleic acid molecules that are separated from other nucleic acid molecules present in the natural source of the nucleic acid. For example, an isolated nucleic acid can be at least 10, 20, 40, 50, 60, 70, 80, or 90% pure, e.g., more than 99% pure. For example, with regards to genomic DNA, the term “isolated” includes nucleic acid molecules which are separated from the chromosome with which the genomic DNA is naturally associated. In some embodiments, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Examples of flanking sequences include adjacent genes, transposons, and regulatory sequences. Moreover, an “isolated” nucleic acid molecule, such as a cDNA molecule, can be substantially free of other cellular material, of culture medium when produced by recombinant techniques, or of chemical precursors or other chemicals when chemically synthesized.

As used herein, the term “hybridizes under low stringency, medium stringency, high stringency, or very high stringency conditions” describes conditions liar hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. Specific hybridization conditions referred to herein are as follows: 1) low stringency hybridization conditions in 6×sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C. followed by one or more washes in 0.2×SSC, 0.1% SDS at 60° C.; 3) high stringency hybridization conditions in 6×SSC at about 45° C., followed by one or more washes in 0.2×SSC, 0.1% SDS at 6.5° C.; and preferably 4) very high stringency hybridization conditions are 0.5 M sodium phosphate, 7% SDS at 65° C., followed by one or more washes at 0.2×SSC, 1% SDS at 65° C. Very high stringency conditions (4) are the preferred conditions and the ones that should be used unless otherwise specified. Methods of the invention can include use of an isolated nucleic acid molecule of the invention that hybridizes under a stringency condition described herein to a sequence described herein or use of a polypeptide encoded by such a sequence, e.g., the molecule can be a naturally occurring variant.

As used herein, a “naturally-occurring” nucleic acid molecule refers to an RNA or DNA molecule having a nucleotide sequence that occurs in Nature. For example, a naturally occurring nucleic acid molecule can encode a natural protein.

As used herein, the terms “gene” and “recombinant gene” refer to nucleic acid molecules which include at least an open reading frame encoding a protein or subunit, derivative, or functional domain thereof. The gene also includes non-coding sequences, e.g., regulatory sequences (e.g., transcriptional and translational regulatory sequences) and introns. Some regulatory sequences can be quite distant, depending on the gene and, e.g., chromosomal organization.

The term “polypeptide” refers to a polymer of three or more amino acids linked by a peptide bond. The polypeptide may include one or more unnatural amino acids. Typically, the polypeptide includes only natural amino acids. The term “peptide” refers to a polypeptide that is between three and thirty-two amino acids in length. A protein can include one or more polypeptide chains. A polypeptide may include one or more unnatural amino acids. Typically, the polypeptide includes only natural amino acids.

A protein or polypeptide can also include one or more modifications, e.g., a glycosylation, amidation, phosphorylation, and so forth.

An “isolated” or “purified” polypeptide or protein is substantially free of cellular material or other contaminating proteins from the cell or tissue source from which the protein is derived, or substantially free from chemical precursors or other chemicals when chemically synthesized. “Substantially free” means that the protein of interest in the preparation is at least 10% pure. In an embodiment, the preparation of the protein has less than about 30%, 20%, 10% and more preferably 5% (by dry weight), of a contaminating component (e.g., a protein not of interest, chemical precursors, and so forth). When the protein or biologically active portion thereof is recombinantly produced, it is also preferably substantially free of culture medium, ie., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of protein without abolishing or substantially altering activity, e.g., the activity is at least 20%, 40%, 60%, 70% or 80% of wild-type. An “essential” amino acid residue is a residue that, when altered from the wild-type sequence results in abolishing activity such that less than 20% of the wild-type activity is present. Conserved amino acid residues are frequently predicted to be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains(e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a protein is preferably replaced with another amino acid residue from the same side chain family. Alternatively, in another embodiment, mutations can be introduced randomly along all or part of a coding sequence, such as by saturation mutagenesis, and the resultant mutants can be screened for biological activity to identify mutants that retain activity. Following mutagenesis, the encoded protein can be expressed recombinantly and the activity of the protein can be determined.

As used herein, a “biologically active portion” or a “functional domain” of a protein includes a fragment of a protein of interest which participates in an interaction, e.g., an intramolecular or an inter-molecular interaction, e.g., a binding or catalytic interaction. An inter-molecular interaction can be a specific binding interaction or an enzymatic interaction (e.g., the interaction can be transient and a covalent bond is formed or broken). An inter-molecular interaction can be between the protein and another protein, between the protein and another compound, or between a first molecule and a second molecule of the protein (e.g., a dimerization interaction). Biologically active portions/functional domains of a protein include peptides comprising amino acid sequences sufficiently homologous to or derived from the amino acid sequence of the protein which include fewer amino acids than the full length, natural protein, and exhibit at least one activity of the natural protein. Biological active portions/functional domains can be identified by a variety of techniques including truncation analysis, site-directed mutagenesis, and proteolysis. Mutants or proteolytic fragments can be assayed for activity by an appropriate biochemical or biological (e.g., genetic) assay. In some embodiments, a functional domain is independently folded. Typically, biologically active portions comprise a domain or motif with at least one activity of a protein., e.g., SOS1 (also discussed below). A biologically active portion/functional domain of a protein can be a polypeptide which is, for example, 10, 25, 50, 100, 200 or more amino acids in length. Biologically active portions/functional domain of a protein can be used as targets for developing agents which modulate SOS1.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, 50%, 60%, 70%, 80%, 90%, 95% or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using the NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of Meyers and Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

Some polypeptides of the present invention can have an amino acid sequence substantially identical to an amino acid sequence described herein, in the context of an amino acid sequence, the term “substantially identical” is used herein to refer to a first amino acid that contains a sufficient or minimum number of amino acid residues that are i) identical to, or ii) conservative substitutions of aligned ammo acid residues in a second amino acid sequence such that the first and second amino acid sequences can have a common structural domain and/or common functional activity. Methods of the invention can include use of a polypeptide that includes an amino acid sequence that contains a structural domain having at least about 60%, or 65% identity, likely 75% identity, more likely 85%, 90%, 92%, 94%, 95%, 96%, 97%, 98% or 99% identity to a domain of a polypeptide described herein.

In the context of nucleotide sequence, the term “substantially identical” is used herein to refer to a first nucleic acid sequence that contains a sufficient or minimum number of raideotides that are identical to aligned nucleotides in a second nucleic acid sequence such that the first and second nucleotide sequences encode a polypeptide having common functional activity, or encode a common structural polypeptide domain or a common functional polypeptide activity. Methods of the invention can include use of a nucleic acid that includes a region at least about 60%, or 65% identity, likely 75% identity, more likely 85%, 90%, 91%, 92%, 93%, 94%, 95%, 96%, 97%, 98% or 99% identity to a nucleic acid sequence described herein, or use of a protein encoded by such nucleic acid.

A “purified preparation of cells”, as used herein, refers to an in vitro preparation of cells. In the case cells from multicellular organisms (e.g., plants and animals), a purified preparation of cells is a subset of cells obtained from the organism, not the entire intact organism. In the case of unicellular microorganisms (e.g., cultured cells and microbial cells), it consists of a preparation of at least 10% and more preferably 50% of the subject cells.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when. used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “small organic molecule” is an organic molecule of having a molecular weight of less than 5, 2, 1, or 0.5 kDa. In many embodiments, such small molecules do not include a peptide bond or a phosphodiester bond. For example, they can be non-polymeric. In some embodiments, the molecule has a molecular weight of at least 50, 100, 200, or 400 Daltons.

“Binding affinity” refers to the apparent dissociation constant or K_{D}. A ligand may, for example, have a binding affinity of at least 10⁻⁵, 10⁻⁶, 10⁻⁷ or 10⁻⁸ M for a particular target molecule. Higher affinity binding of a ligand to a first target relative to a second target can be indicated by a smaller numerical value K_{D}¹ for binding the first target than the numerical value K_{D}² for binding the second target. In such cases the ligand has specificity for the first target relative to the second target. The agent may bind specifically to the target, e.g., with an affinity that is at least 2, 5, 10, 100, or 1000 better than for a non-target. For example, an agent can bind to SOS1 with a K_{d} of less than 10⁻⁵, 10⁻⁶, 10⁻⁷ or 10⁻⁸ M in PBS.

Binding affinity can be determined by a variety of methods including equilibrium dialysis, equilibrium binding, gel filtration, ELISA, or spectroscopy (e.g., using a fluorescence assay). These techniques can be used to measure the concentration of bound and free ligand as a function of ligand (or target) concentration. The concentration of bound ligand ([Bound]) is related to the concentration of free ligand ([Free]) and the concentration of binding sites for the ligand on the target where (N) is the number of binding sites per target molecule by the following equation:

[Bound]=N·[Free]/(1/Ka)+[Free])

The details of one or more embodiments of the invention are set forth in the accompanying drawings and the description below. Other features, objects, and advantages of the invention will be apparent from the description and drawings, and from the claims. All cited patents, and patent applications and references (including references to public sequence database entries) are incorporated by reference in their entireties for all purposes.

### DESCRIPTION OF DRAWINGS

FIG. 1 SOS1 mutations cause NS. FIG. 1a is a schematic diagram of the genomic organization of the human SOS1 gene and domains within the SOS1 protein product. HF (Histone-like fold domain), DH (Dbl-homology domain), PH (Pleckstrin homology domain), HL (helical linker), REM (RAS-exchange motif), Cdc25 (Cdc25 domain), and poly-proline domains are shown, as are the nucleotide positions and predicted amino acid changes (in single letter amino acid code) of identified NS-associated variants. Whether P655L is a bona fide mutation or a rare polymorphism is unclear; see text for details. FIGS. 1b-d are representative chromatograms showing SOS1 mutations in NS patients. T266K (FIG. 1b), M269R (FIG. 1c), E846K (FIG. 1d),

FIG. 2 is a table listing genetic and clinical features of SOS1 mutation probands.

FIG. 3 is a table listing genotype-phenotype correlations in Noonan syndrome.

FIG. 4 Position of NS mutations on SOS1 structure. NS mutations were mapped onto the crystal structure of the SOS1 DH, PH, HL and Cdc25 domains (PDB 1XD4) or in FIG. 4d, the REM/Cdc25 domain/RAS co-crystal (PDB 1NVV), using PyMo1 (available on the World Wide Web at pymol.sourceforge.net). FIG. 4a is a schematic diagram showing positions of all NS mutants, as well as the predicted locations of HF (PDB 1Q9C) and allosteric (arrows) and effector (arrowhead) Ras binding. FIG. 4b is a cartoon illustrating proposed regulation of SOS1 Ras-GEF activity by its N-terminal domains; A, allosteric Ras; E, effector Ras. FIGS. 4c-h are schematic diagrams showing the locations of NS mutants. FIGS. 4c-e show the region of SOS1 containing M269R and T266K. FIG. 4c, WT SOS1, with position of M269 indicated (arrow). FIG. 4d, WT RAS/SOS1 co-crystal (PDB 1NVV) structure, showing RAS I31 binding to the hydrophobic pocket in the REM domain (arrow). FIG. 4e, Model of M269R mutant. M269R should lead to loss of contact between the DH and REM domains because substitution of a larger, charged arginine for the smaller hydrophobic methionyl is predicted to result in a collision (arrow) with L678 in the REM domain. FIGS. 4f, g, Position and predicted consequences of Y337C mutation. FIG. 4f, In WT SOS1, Y337, which lies within the DH domain, participates in a hydrophobic interaction with M538 (yellow arrow) and hydrogen bonds with N502 in the PH domain (green arrow); both of these interactions are predicted to be lost in Y337C (FIG. 4g). FIG. 4h, E846K mutant, The Cdc25 domain residue E846 (upper left panel) normally forms a salt bridge with K1029 (arrow) within an extended loop that traverses the distal end of the Cdc25 domain (right panel). Mutation of E846 to lysine should disrupt this interaction, and result in electrostatic repulsion between the E846K and 1029K and displacement of the loop from its normal position.

FIG. 5 NS-associated SOS1 mutations are gain-of-function mutants. FIG. 5a, SOS1 mutants cause sustained ERK activation. The indicated SOS1 expression constructs were co-transfected with an HA-ERK1 expression vector into 293T cells. Transfected cells were starved and stimulated with EGF (20 ng/ml) as described in Methods, and lysates were subjected to immunoblotting with anti-pERK antibodies, followed by re-probing with anti-HA antibodies to control for loading and anti-SOS1 antibodies to assess the level of SOS1 expression. Under these conditions, transfection efficiency is ˜50% (as estimated by using an expression construct for green fluorescent protein). Accordingly, based on SOS1 levels in the Immunoblots, we estimate that WT SOS1 and the NS mutants are expressed to 3-4 higher levels than endogenous SOS1 on a per-cell basis. Each mutant was analyzed in at least three separate transfection. The upper panels are representative experiments. The lower panels show quantification of ERK activation at 15 minutes post-stimulation in data pooled from 3 separate experiments, shown as fold-activation compared to cells transfected with wild type SOS1 (WT). Error bars represent standard deviations. *, P<0.01 and #, P<0.05 compared to ERK activation by WE SOS1 (two-tailed Student's t test). None of the mutants caused a significant difference in ERK activation at 5 minutes post-stimulation. FIG. 5b, SOS1 mutants enhance EGF-evoked RAS activation. WT SOS1 and the indicated mutants were over-expressed in 293T cells, and EGF-stimulated endogenous RAS activation was assessed by the GST-RAF-RBD binding assay. The left panel shows a representative time course experiment (from 3 independent determinations). The right hand panel shows quantification of RAS-GTP at 15 minutes post-stimulation, compared to vector alone, *, P<0.05 compared to WT SOS1 (1-tailed Student's t-test), M269R also caused significantly increased RAS activation at 5 minutes post-FOE addition. FIG. 5e, NS and CFC mutations affect RAS/ERK pathway at different levels. A simplified schematic of the RAS/ERK pathway, with positions of mutant genes associated with NS and CFC shown. *indicates that HRas mutations cause Costello Syndrome; **indicates that Nfl imitations are the cause of neurofibromatosis, Type I. Note that NS mutations affect the upstream components, whereas CFC mutations involve more distal signaling molecules. The relative positions of these mutants probably accounts for the phenotypic overlap and differences between these syndromes.

FIGS. 6A-6D are diagrams depicting the positions of cancer-associated mutations in the SOS1 polypeptide structure.

Like reference symbols in the various drawings indicate like elements.

### DETAILED DESCRIPTION

The SOS1 gene encodes a developmentally essential Ras guanine nucleotide exchange factor (Ras-GEF). As described in more detail below, we discovered missense mutations in SOS1 in ˜20% (12/59) of NS cases not associated with a PTPN11 mutation, NS patients with SOS1 mutations were more likely to have pulmonic stenosis (PS) than those with neither a SOS1 nor a PTPN11 mutation, whereas atrial septal defect (ASD) was less common in NS patients with SOS1 mutations than in those with mutant PTPN11 alleles. We also discovered that SOS1 mutation-associated NS has a different prevalence of pulmonic stenosis and atrial septal defects than NS caused by other genes. NS-associated SOS1 mutations are hypermorphs whose products enhance RAS and ERK activation. Our results identify SOS1 mutants as a major cause of NS and represent the first example of GEF mutations associated with human disease.

The genes known to cause NS and the phenotypically related Cardio-facial-cutaneous syndrome (CFC) encode members of the RAS/ERK pathway (Bentires-Alj et al., Nature Med., 12:11-13, 2006). RAS genes (KRAS, HRAS, NRAS) encode small GTP-binding (small G) proteins that act as molecular switches. In their GDP-bound state (RAS-GDP), RAS proteins are inactive. Cell stimulation (e.g., by growth factors or cytokines) results in exchange of GTP for GDP, a process catalyzed by RAS-GEFs. GTP-bound RAS proteins (RAS-GTP) bind and promote the activation of several downstream effectors, including RAF family serine-threonine kinases (cRAF, BRAF, A-RAF), phosphatidylinositol 3-kinase, and RAL-guanine nucleotide dissociation stimulator (RAL-GDS). Activated RAF phosphorylates and activates MEK1/2, which phosphorylate and activate ERK1/2. RAS proteins also have an intrinsic GTPase activity, which, aided by RAS-GTPase-activating proteins (RAS-GAPs), restores RAS-GTP to the inactive, GTP-bound state. Mutations in PTPN11 and KRAS cause NS, whereas mutations in BRAF, MEK1 or MEK2 cause CFC, and the common features of both disorders appear to be the result of increased ERK activation (Niihori et al., Nat. Genet., 38:294-296, 2006: Rodriguez-Viciana et al., Science, 311:1287-1290, 2006).

Because NS and CFC are clinically distinguishable, however, and CFC is caused by mutations in genes acting downstream in the RAS/ERK pathway, we investigated more upstream components as candidate NS genes.

### SOS1

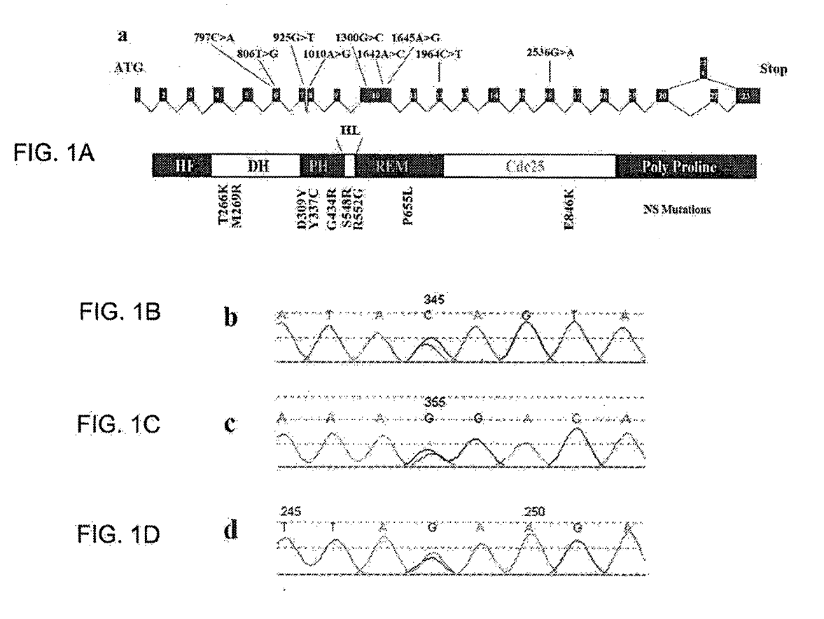

The human SOS1 gene, located on chromosome 2p, spans 136 kb and has 23 coding exons, with the open reading frame beginning in exon 1 and terminating in exon 23 (FIG. 1a). Alternative splicing of exon 22 results in two isoforms that differ in Grb2 binding affinity and biological activity (Rojas et al., Oncogene, 12, 2291-2300, 1996). The translation initiation codon is located at nucleotides 45 to 47 of exon 2. The open reading frame is terminated at nucleotide 492 in exon 24. The open reading frame contains 4,002 nucleotides and encodes 1,333 amino acids. The coding sequence of wild type human SOS1 is shown in Table A (see also GenBank® Acc. No. NM_{—}005633, GI:15529995).

TABLE A
Human SOS1 coding sequence

ATGCAGGCGCAGCAGCTGCCCTACGAGTTTTTCAGCGAAGAGAACGCGCC
CAAGTGGCGGGGACTACTGGTGCCTGCGCTGAAAAAGGTCCAGGGGCAAG
TTCATCCTACTCTCGAGTCTAATGATGATGCTCTTCAGTATGTTGAAGAA
TTAATTTTGCAATTATTAAATATGCTATGCCAAGCTCAGCCCCGAAGTGC
TTCAGATGTAGAGGAACGTGTTCAAAAAAGTTTCCCTCATCCAATTGATA
AATGGGCAATAGCTGATGCCCAATCAGCTATTGAAAAGAGGAAGCGAAGA
AACCCTTTATCTCTCCCAGTAGAAAAAATTCATCCTTTATTAAAGGAGGT
CCTAGGTTATAAAATTGACCACCAGGTTTCTGTTTACATAGTAGCAGTCT
TAGAATACATTTCTGCAGACATTTTAAAGCTGGTTGGGAATTATGTAAGA
AATATACGGCATTATGAAATTACAAAACAAGATATTAAAGTGGCAATGTG
TGCTGACAAGGTATTGATGGATATGTTTCATCAAGATGTAGAAGATATTA
ATATATTATCTTTAACTGACGAAGAGCCTTCCACCTCAGGAGAACAAACT
TACTATGATTTGGTAAAAGCATTTATGGCAGAAATTCGACAATATATAAG
GGAACTAAATCTAATTATAAAAGTTTTTAGAGAGCCCTTTGTCTCCAATT
CAAAATTGTTTTCAGCTAATGATGTAGAAAATATATTTAGTCGCATAGTA
GATATACATGAACTTAGTGTAAAGTTACTGGGCCATATAGAAGATACAGT
AGAAATGACAGATGAAGGCAGTCCCCATCCACTAGTAGGAAGCTGCTTTG
AAGACTTAGCAGAGGAACTGGCATTTGATCCATATGAATCGTATGCTCGA
GATATTTTGCGACCTGGTTTTCATGATCGTTTCCTTAGTCAGTTATCAAA
GCCTGGGGCAGCACTTTATTTGCAGTCAATAGGCGAAGGTTTCAAAGAAG
CTGTTCAATATGTTTTACCCAGGCTGCTTCTGGCCCCTGTTTACCACTGT
CTCCATTACTTTGAACTTTTGAAGCAGTTAGAAGAAAAAAGTGAAGATCA
AGAAGACAAGGAATGTTTAAAACAAGCAATAACAGCTTTGCTTAATGTTC
AGAGTGGTATGGAAAAAATATGTTCTAAAAGTCTTGCAAAACGAAGACTG
AGTGAATCTGCATGTCGGTTTTATAGTCAGCAAATGAAGGGGAAACAACT
AGCAATCAAGAAGATGAACGAGATTCAGAAGAATATTGATGGTTGGGAGG
GAAAAGACATTGGACAGTGTTGTAATGAATTTATAATGGAAGGAACTCTT
ACACGTGTAGGAGCCAAACATGAGAGACACATATTTCTCTTTGATGGCTT
AATGATTTGCTGTAAATCAAATCATGGGCAGCCAAGACTTCCTGGTGCTA
GCAATGCAGAATATCGTCTTAAAGAAAAGTTTTTTATGCGAAAGGTACAA
ATTAATGATAAAGATGACACCAATGAATACAAGCATGCTTTTGAAATAAT
TTTAAAAGATGAAAATAGTGTTATATTTTCTGCCAAGTCAGTGAAAGAGA
AAAACAATTGGATGGCAGCATTGATATCTTTACAGTACCGGAGTACACTG
GAAAGGATGCTTGATGTAACAATGCTACAGGAAGAGAAAGAGGAGCAGAT
GAGGCTGCCTAGTGCTGATGTTTATAGATTTGCAGAGCCTGACTCTGAAG
AGAATATTATATTTGAAGAGAACATGCAGCCCAAGGCTGGAATTCCAATT
ATCAAAGCAGGAACTGTTATTAAACTTATAGAGAGGCTTACGTACCATAT
GTACGCAGATCCCAATTTTGTTCGGACATTTCTTACAACATACAGATCCT
TTTGCAAACCTCAAGAACTACTGAGTCTTATAATAGAAAGGTTTGAAATT
CCAGAGCCTGAGCCAACAGAAGCTGATCGCATAGCTATAGAGAATGGAGA
TCAACCCTTGAGTGCAGAACTGAAAAGATTTAGAAAAGAATATATACAGC
CTGTGCAACTGCGAGTATTAAATGTATGTCGGCACTGGGTAGAGCACCAC
TTCTATGATTTTGAAAGAGATGCATATCTTTTGCAACGAATGGAAGAATT
TATTGGAACAGTAAGAGGTAAAGCAATGAAAAAATGGGTTGAATCCATCA
CTAAAATAATCCAAAGGAAAAAAATTGCAAGAGACAATGGACCAGGTCAT
AATATTACATTTCAGAGTTCACCTCCCACAGTTGAGTGGCATATAAGCAG
ACCTGGGCACATAGAGACTTTTGACCTGCTCACCTTACACCCAATAGAAA
TTGCTCGACAACTCACTTTACTTGAATCAGATCTATACCGAGCTGTACAG
CCATCAGAATTAGTTGGAAGTGTGTGGACAAAAGAAGACAAAGAAATTAA
CTCTCCTAATCTTCTGAAAATGATTCGACATACCACCAACCTCACTCTGT
GGTTTGAGAAATGTATTGTAGAAACTGAAAATTTAGAAGAAAGAGTAGCT
GTGGTGAGTCGAATTATTGAGATTCTACAAGTCTTTCAAGAGTTGAACAA
CTTTAATGGTGTCCTTGAGGTTGTCAGTGCTATGAATTCATCACCTGTTT
ACAGACTAGACCACACATTTGAGCAAATACCAAGTCGCCAGAAGAAAATT
TTAGAAGAAGCTCATGAATTGAGTGAAGATCACTATAAGAAATATTTGGC
AAAACTCAGGTCTATTAATCCACCATGTGTGCCTTTCTTTGGAATTTATC
TCACTAATATCTTGAAAACAGAAGAAGGCAACCCTGAGGTCCTAAAAAGA
CATGGAAAAGAGCTTATAAACTTTAGCAAAAGGAGGAAAGTAGCAGAAAT
AACAGGAGAGATCCAGCAGTACCAAAATCAGCCTTACTGTTTACGAGTAG
AATCAGATATCAAAAGGTTCTTTGAAAACTTGAATCCGATGGGAAATAGC
ATGGAGAAGGAATTTACAGATTATCTTTTCAACAAATCCCTAGAAATAGA
ACCACGAAACCCTAAGCCTCTCCCAAGATTTCCAAAAAAATATAGCTATC
CCCTAAAATCTCCTGGTGTTCGTCCATCAAACCCAAGACCAGGTACCATG
AGGCATCCCACACCTCTGCAGCAGGAGCCAAGGAAAATTAGTTATAGTAG
GATCCCTGAAAGTGAAACAGAAAGTACAGCATCTGCACCAAATTCTCCAA
GAACACCGTTAACACCTCCGCCTGCTTCTGGTGCTTCCAGTACCACAGAT
GTTTGCAGTGTATTTGATTCCGATCATTCGAGCCCTTTTCACTCAAGCAA
TGATACCGTCTTTATCCAAGTTACTCTGCCCCATGGCCCAAGATCTGCTT
CTGTATCATCTATAAGTTTAACCAAAGGCACTGATGAAGTGCCTGTCCCT
CCTCCTGTTCCTCCACGAAGACGACCAGAATCTGCCCCAGCAGAATCTTC
ACCATCTAAGATTATGTCTAAGCATTTGGACAGTCCCCCAGCCATTCCTC
CTAGGCAACCCACATCAAAAGCCTATTCACCACGATATTCAATATCAGAC
CGGACCTCTATCTCAGACCCTCCTGAAAGCCCTCCCTTATTACCACCACG
AGAACCTGTGAGGACACCTGATGTTTTCTCAAGCTCACCACTACATCTCC
AACCTCCCCCTTTGGGCAAAAAAAGTGACCATGGCAATGCCTTCTTCCCA
AACAGCCCTTCCCCCTTTACACCACCTCCTCCTCAAACACCTTCTCCTCA
CGGCACAAGAAGGCATCTGCCATCACCACCATTGACACAAGAAGTGGACC
TTCATTCCATTGCTGGGCCGCCTGTTCCTCCACGACAAAGCACTTCTCAA
CATATCCCTAAACTCCCTCCAAAAACTTACAAAAGGGAGCACACACACCC
ATCCATGCACAGAGATGGACCACCACTGTTGGAGAATGCCCATTCTTCCT
GA
(SEQ ID NO: 1)

The amino acid sequence of wild type human SOS1 polypeptide is shown in Table B (see also GenBank® Acc. No. NP_{—}005624, GI:15529996).

TABLE B
Human SOS1 amino acid sequenec

MQAQQLPYEFFSEENAPKWRGLLVPALKKVQGQVHPTLESSNDDALQYVE
ELILQLLNMLCQAQPRSASDVEERVQKSFPHPIDKWAIADAQSAIEKRKR
RNPLSLPVEKIHPLLKEVLGYKIDHQVSVYIVAVLEYISADILKLVGNYV
RNIRHYEITKQDIKVAMCADKVLMDMFHQDVEDINILSLTDEEPSTSGEQ
TYYDLVKAFMAEIRQYIRELNLIIKVFREPFVSNSKLFSANDVENIFSRI
VDIHELSVKLLGHIEDTVEMTDESGPHPLVGSCFEDLAEELAFDPYESYA
RDILRPGFHDRFLSQLSKPGAALYLQSIGEGFKEAVQTVLPRLLLAPVYH
CLHTPELLKQLEEKSEDQEDKECLKQAITALLNVQSGMEKICSKSLAKRR
LSESACRFYSQQMKGKQLAIKKMNEIQKNIDGWEGKDIGQCCNEFIMEGT
LTRVGAKHERHIFLFDGLMICCKSNHGQPRLPGASNAEYRLKEKFFMRKV
QINDKDDTNEYKHAFEIILKDENSVIFSAKSAEEKNNWMAALISLQYRST
LERMLDVTMLQEEKEEQMRLPSADVYRFAEPDSEENIIFEENMQPKAGIP
IIKAGTVIKLIERLTYHMTADPNFVRTFLTTYRSFCKPQELLSLIIERFE
IPEPEPTEADRIAIENGDQPLSAELKRFRKEYIQPVQLRVLNVCRHWVEH
HFYDFERDAYLLQRMEEFIGTVRGKAMKKWVESITKIIQRKKIARDNGPG
HNITFQSSPPTVEWHISRPGHIETFDLLTLHPIEIARQLTLLESDLYRAV
QPSELVGSVWTKEDKEINSPNLLKMTRHTTNLTLWFEKCIVETENLEERV
AVVSRIIEILQVFQELNNFNGVLEVVSAMNSSPVYRLDHTFEQIPSRQKK
ILEEAHELSEDHYKKYLAKLRSINPPCVPFFGIYLTNILKTEEGNPEVLK
RHGKELINFSKRRKVAEITGEIQQYQNQPYCLRVESDIKRFFENLNPMGN
SMEKEFTDYLFNKSLEIEPRNPKPLRFPKKYSYPLKSPGVRPSNPRPGTM
RHPTPLQQEPRKISYSRIPESETESTASAPNSPRTPLTPPPASGASSTTD
VCSVFDSDHSSPFHSSNDTVFIQVTLPHGRPSASVSSISLTKGTDEVPVP
PPVPPRRRPESAPAESSPSKIMSKHLDSPPAIPPRQPTSKAYSPRYSISD
RTSISDPPESPPLLPPREPVRTPDVFSSSPLHLQPPPLGKKSDHGNAFFP
NSPSPFTPPPPQTPSPHGTRRHLPSPPLTQEVDLHSIAGPPVPPRQSTSQ
HIPKLPPKTYKREHTHPSMHRDGPPLLENAHSS
(SEQ ID NO: 2)

The SOS1 polypeptide is a guanine nucleotide exchange factor for Ras and interacts with growth factor receptor-bound protein 2 (GRB2). In addition to its Cdc25 (Ras-exchange) domain, Sos-1 contains several other highly conserved domains, including a Histone-like Fold (HF), followed by Dbl Homology (DH) and Pleckstrin Homology (PH) domains, a Helical Linker (HL), a Ras Exchange Motif (REM), and a proline-rich region (FIG. 1A). The locations of the domains in the SOS1 polypeptide are as follows: the DH domain is at amino acids 198-404, the PH domain is at amino acids 418-547, the HL domain is at amino acids 548-563, the Ras-exchange motif REM is at amino acids 576-740, the cdc25 domain is at amino acids 750-1040, and the PXXP domain is 1050 to 1300. The PXXP domain mediates binding to SH3 domains.

Evaluation of SOS1-related genes and polypeptides, as well as genes and gene products implicated in a SOS signaling pathway, is also contemplated. For example, the presence of mutations in SOS2 genes and gene products may be determined. A human SOS2 coding sequence is listed under GenBank® Acc. no. NM_{—}006939.1, GI:39930603. The corresponding polypeptide sequence is listed under GenBank® Acc. no. NP_{—}008870.1, GI:39930604.

### Mutant SOS1 Nucleic Acid Molecules and Polypeptides

One aspect of the invention features isolated nucleic acid molecules that encode a mutant SOS1 polypeptide and that are associated with NS and/or a neoplastic disorder. The invention features nucleic acid molecules that encode mutant SOS1 polypeptides (“mtSOS1”) or biologically active portions thereof, as well as, nucleic acid fragments sufficient for use as hybridization probes to identify a mutant SOS1-encoding nucleic acid (e.g., mtSOS1 mRNA) and mutants thereof.

A nucleic acid molecule for use in the present invention, e.g., a nucleic acid molecule having the nucleotide sequence of SEQ ID NO:1 with at least one nucleotide mutation (e.g., with 1, 2, 3, 4, 5, 10, or 20 nucleotide mutations), or a portion thereof, can be isolated using standard molecular biology techniques and the sequence information provided herein. For example, a human SOS1 cDNA with a mutation can be generated by introducing the mutation into a wild type SOS1 cDNA (e.g., using site-directed mutagenesis or other recombinant means). Alternatively, a mutant SOS1 cDNA can be isolated from a cDNA library derived from an individual with NS using all or portion of SEQ ID NO:1 as a hybridization probe and standard hybridization techniques (e.g., as described in Sambrook, J. Fritsh, E. F., and Maniatis, T. Molecular Cloning: A Laboratory Manual 2nd, ed, Cold Spring Harbor Laboratory, Cold Spring Harbor Laboratory Press, Cold Spring Harbor, N.Y., 1989).

In one embodiment, an isolated nucleic acid molecule for use in methods described herein includes a mutation which results in an amino acid change. The sequence of SEQ ID NO:1 corresponds to the wild type human SOS1 cDNA. In various embodiments, the mtSOS1 cDNA includes a mutation at one of the following positions of the SEQ ID NO:1: 797, 806, 925, 1010, 1358, 1642, 1654, 1964, or 2536.

In another preferred embodiment, an isolated nucleic acid molecule for use in the invention includes a nucleic acid molecule which is a complement of the nucleotide sequence shown in SEQ ID NO:1 with at least one nucleotide mutation. A nucleic acid molecule which is complementary to the nucleotide sequence shown in SEQ ID NO:1, or mutant sequence thereof, is one which is sufficiently complementary to the nucleotide sequence such that it can hybridize to the nucleotide sequence, thereby forming a stable duplex.

Moreover, the nucleic acid molecule for use in the invention earl include only a portion of a mutant SOS1 sequence (e.g., a portion of SEQ ID NO:1 containing a mutation), for example, a fragment which can be used as a probe or primer or a fragment encoding a biologically active portion of a mutant form of SOS1. The nucleotide sequence determined from analyzing the sequence of a SOS1 gene from individuals with NS allows for the generation of probes and primers designed for use in identifying individuals with NS and neoplastic disorders. The primers can also be used to clone and/or sequence mtSOS1 homologues (e.g., missense homologues), as well as mtSOS1 homologues from other organisms. The probe/primer typically includes substantially purified oligonucleotide. The oligonucleotide typically includes a region of nucleotide sequence that hybridizes under stringent conditions to at least about 12, preferably about 25, more preferably about 40, 50 or 75 consecutive nucleotides of SEQ ID NO:1 or the complementary sequence thereof. Primers which hybridize to non-coding regions of the SOS1 gene are also contemplated. Primers (e.g., primers based on the nucleotide sequence in SEQ ID NO:1 or on non-coding SOS1 sequence) can be used in PCR reactions to identify if an individual has a mutation. Probes based on the SOS1 nucleotide sequences can be used to detect transcripts or genomic sequences encoding the mtSOS1 protein. In various embodiments, the probe further includes a label group attached thereto, e.g. the label group can be a radioisotope, a fluorescent compound, an enzyme, or am enzyme co-factor. Such probes can be used as a part of a diagnostic test kit for: identifying cells or tissue which express a mtSOS1 protein, detecting the levels of SOS1 mRNA or determining whether a genomic SOS1 gene is mutated or deleted. For example, primers or probes can also be used in diagnostic screening to identify individuals suffering from NS or a neoplastic disorder.

Kits for diagnosing patients affected with NS or a neoplastic disorder are provided. For example, the kit can include a probe or a primer, e.g., a labeled or labelable probe or primer, capable of detecting a genetic lesion, e.g., a point mutation, in a SOS1 gene or a product of the gene (e.g., an mRNA or cDNA). The primer can be packaged in a suitable container. The kit can also include reagents required for PCR amplification and/or DNA sequencing. The kit can further include instructions for using the kit to diagnose NS, other cardiovascular or growth related disorders, or a neoplastic disorder.

In one embodiment, the nucleic acid molecule of the invention encodes a protein or portion thereof which includes an amino acid sequence which is at least 70%, 80%, 90%, 95%, 96%, 97%, 98%, or 99% identical to SEQ ID NO:2, or a portion thereof, and which contains at least one amino acid change relative to SEQ ID NO:2, or the portion thereof. The protein exhibits activities which are the same as, or enhanced relative to, a wild type SOS1 protein.

Portions of proteins encoded by the mtSOS1 nucleic acid molecule are preferably biologically active portions of the SOS1 protein which play a role in signaling, e.g., domains required for SOS1-mediated Ras-MAPK activation, such as the cdc25 domain.

In another embodiment, an isolated nucleic acid molecule of the invention is at least 15 nucleotides in length and hybridizes under stringent conditions to the nucleic acid molecule comprising the nucleotide sequence of SEQ ID NO:1 or to a mutant sequence thereof. In other embodiments, the nucleic acid is at least 30, 50, 100, 205, 210, 220, 230, 250, 300, 400, 500, or 600 nucleotides in length. A “wild type” sequence refers to a sequence that is a naturally-occurring, normal, non-mutated version of the sequence. A wild type sequence is not associated with NS or a neoplastic disorder.

Another aspect of the invention features isolated SOS1 proteins, and biologically active portions thereof, as well as peptide fragments suitable for use as immunogens to raise anti-SOS1 or mtSOS1 antibodies.

In one embodiment, the mtSOS1 protein or portion thereof includes an amino acid sequence which is at least 70%, 80%, 90%, 95%, 96%, 97%, 98%, or 99% identical to SEQ ID NO:2, or a portion thereof, and which contains at least one amino acid change relative to SEQ ID NO:2. The protein exhibits activities which are the same as, or enhanced relative to, a wild type SOS1 protein. The portion of the protein is preferably a biologically active portion as described herein. The invention also provides mtSOS1 chimeric or fusion proteins.

An isolated mtSOS1 protein, or portions or fragments thereof, can be used as an immunogen to generate antibodies that bind mtSOS1 using standard techniques for polyclonal and monoclonal antibody preparation. The antigenic peptide of mtSOS1 includes at least 8 amino acid residues of the amino acid sequence shown in SEQ ID NO:2, containing a mutation, and encompasses an epitope of mtSOS1 such that an antibody raised against the peptide forms a specific immune complex with the mtSOS1 protein. Preferably, the antigenic peptide includes at least 10 amino acid residues, more preferably at least 15 amino acid residues, even more preferably at least 20 amino acid residues, and most preferably at least 30 amino acid residues. Preferred epitopes encompassed by the antigenic peptide are hydrophilic regions.

In another aspect of the invention features anti-SOS1 or anti-mtSOS1 antibodies. The term “antibody” as used herein refers to immunoglobulin molecules and immunologically active portions of immunoglobulin molecules, i.e., molecules that contain an antigen binding site which specifically binds (immunoreacts with) an antigen, such as SOS1 or mtSOS1. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind SOS1 or mtSOS1. The term “monoclonal antibody” or “monoclonal antibody composition”, as used herein, refers to a population of antibody molecules that contain only one species of an antigen binding site capable of immunoreacting with a particular epitope of SOS1 or mtSOS1. A monoclonal antibody composition thus typically displays a single binding affinity for a particular SOS1 protein with which it immunoreacts. An anti-SOS1 antibody or anti-mtSOS1 (e.g., monoclonal antibody) can be used to isolate wild type and mutant SOS1 proteins, respectively.

### Evaluating SOS1 Nucleic Acids and Polypeptides

Various methods described herein include steps in which genetic information of a subject is evaluated, e.g., to determine whether the subject has a mutation in a SOS1 gene. SOS1 genetic information can be obtained, e.g., by evaluating genetic material (e.g., DNA or RNA) or proteins from a subject (e.g., as described below). Genetic information can include, for example, an indication about the presence or absence of a particular mutation, e.g., one or more nucleotide insertions, deletions, or substitutions in a SOS1 gene of a subject, or chromosomal rearrangements, alterations in the level of mRNA, splicing, or in a post-translational modification of a polypeptide encoded by the SOS1 gene. Somatic and germ line mutations can be evaluated.

Numerous methods are suitable for evaluating genetic material. These methods can be used to evaluate a SOS1 locus as well as other loci (e.g., other loci associated with Noonan syndrome and/or a neoplastic disorder).

Nucleic acid samples can analyzed using biophysical techniques (e.g., hybridization, electrophoresis, and so forth), sequencing, enzyme-based techniques, and combinations-thereof. For example, hybridization of sample nucleic acids to nucleic acid microarrays can be used to evaluate sequences (e.g., sequences in an mRNA population or in genomic DNA) and to evaluate genetic mutations. Other hybridization based techniques include sequence specific primer binding (e.g., PCR or LCR); Southern analysis of DNA, e.g., genomic DNA; Northern analysis of RNA, e.g., mRNA; fluorescent probe based techniques (see, e.g., Beaudet et al. (2001) Genome Res. 11(4):600-8); and allele specific amplification. Enzymatic techniques include restriction enzyme digestion; sequencing; and single base extension (SBE). These and other techniques are well known to those skilled in the art.

Electrophoretic techniques include capillary electrophoresis and Single-Strand Conformation Polymorphism (SSCP) detection (see, e.g., Myers et al. (1985) Nature 313:495-8 and Ganguly (2002) Hum. Mutat. 19(4):334-42). Other biophysical methods include denaturing high pressure liquid chromatography (DHPLC).

In one embodiment, allele specific amplification technology that depends on selective PCR amplification may be used to obtain genetic information. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition, it is possible to introduce a restriction site in the region of the mutation to create cleavage-based detection (Gasparini et al. (1992) Mol. Cell Probes 6:1). In another embodiment, amplification can be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Enzymatic methods for detecting sequences include amplification based-methods such as the polymerase chain reaction (PCR; Saiki, et al. (1985) Science 230, 1350-1354) and ligase chain reaction (LCR; Wu. et al. (1989) Genomics 4, 560-569; Barringer et al. (1990). Gene 1989, 117-122; F. Barany. 1991, Proc. Natl. Acad. Sci. USA 1988, 189-193); transcription-based methods utilize RNA synthesis by RNA polymerases to amplify nucleic acid (U.S. Pat. No. 6,066,457; U.S. Pat. No. 6,132,997; U.S. Pat. No. 5,716,785; Sarkar et al., Science (1989) 244:331-34; Stofler et al., Science (1988) 239;491); NASBA (U.S. Pat. Nos. 5,130,238; 5,409,818; and 5,554,517); rolling circle amplification (RCA; U.S. Pat. Nos. 5,854,033 and 6,143,495) and strand displacement amplification (SDA; U.S. Pat. Nos. 5,455,166 and 5,624,825). Amplification methods can be used in combination with other techniques.

Other enzymatic techniques include sequencing polymerases, e.g., DNA polymerases and variations thereof such as single base extension technology. See, e.g., U.S. Pat. No. 6,294,336; U.S. Pat. No. 6,013,431; and U.S. Pat. No. 5,952,174

Mass spectroscopy (e.g., MALDI-TOF mass spectroscopy) can be used to detect nucleic acid mutations. In one embodiment, (e.g., the MassEXTEND™ assay, SEQUENOM, Inc.), selected nucleotide mixtures, missing at least one dNTP and including a single ddNTP is used to extend a primer that hybridizes near a mutation. The nucleotide mixture is selected so that the extension products between the different polymorphisms at the site create the greatest difference in molecular size. The extension reaction is placed on a plate for mass spectroscopy analysis.

Fluorescence based detection can also be used to detect nucleic acid mutations. For example, different terminator ddNTPs can be labeled with different fluorescent dyes. A primer can be annealed near or immediately adjacent to a mutation, and the nucleotide at the mutation site can be detected by the type (e.g., “color”) of the fluorescent dye that is incorporated.

Hybridization to microarrays can also be used to detect mutations. For example, a set of different oligonucleotides, with the mutant nucleotide at varying positions with the oligonucleotides can be positioned on a nucleic acid array. The extent of hybridization as a function of position and hybridization to oligonucleotides specific for the other allele can be used to determine whether a particular mutation is present. See, e.g., U.S. Pat. No. 6,066,454.

In one implementation, hybridization probes can include one or more additional mismatches to destabilize duplex formation and sensitize the assay. The mismatch may be directly adjacent to the query position, or within 10, 7, 5, 4, 3, or 2 nucleotides of the query position. Hybridization probes can also be selected to have a particular Tₘ, e.g., between 45-60° C., 55-65° C., or 60-75° C. In a multiplex assay, Tₘ's can be selected to be within 5, 3, or 2° C. of each other.

It is also possible to directly sequence the nucleic acid for a particular genetic locus, e.g., by amplification and sequencing, or amplification, cloning and sequence. High throughput automated (e.g., capillary or microchip based) sequencing apparati can be used. Nucleic acid analysis include sequencing with a pyrophosphate DNA sequencer (454 Life Sciences, New Haven, Conn.; see U.S. Pat. Pub. No. 20050130173) or optical sequencing see, e.g., U.S. Pat. Pub. Nos. 20060024711, 20060136144, and 20060012793).

In still other embodiments, the sequence of a protein of interest is analyzed to infer its genetic sequence. Methods of analyzing a protein sequence include protein sequencing, mass spectroscopy, sequence/epitope specific immunoglobulins, and protease digestion.

Various assays may be used to determine whether a mutant SOS1 polypeptide has increased activity (e.g., enhanced Ras or Erk activation) as compared to a wild type SOS1 polypeptide. For example, SOS1 polypeptides may be expressed in cells under conditions in which Ras or Erk activation can be detected and measured to determine whether expression of at mutant SOS1 polypeptide stimulates enhanced Ras/Erk activation relative to the wild type SOS1 polypeptide. In one exemplary method, DNA encoding the SOS1 polypeptide of interest is co-transfected into cells with a DNA encoding a tagged Erk polypeptide. Cells are stimulated with a cytokine or growth factor that stimulates a receptor tyrosine kinase EGF), Erk activation is detected by immunoblotting the cell lysates to detect phosphorylation of tagged Erk, and activation in the presence of a mutant SOS1 polypeptide is compared to activation in the presence of a wild type SOS1 polypeptide (described further in the Examples, below). Ras activation can be determined, e.g., by precipitating Ras-GTP from growth factor-stimulated cells transfected with mutant or wild type SOS1 and quantitating relative levels of Ras-GTP in the cells. Ras-GTP can be precipitated using a fusion protein consisting of glutathione-S-transferase and the Ras binding, domain (RBD) of Raf (Taylor and Shalloway, Curr. Biol., 6:1621-1627, 1996). SOS1-mediated stimulation of Ras can also be measured using a Ras nucleotide exchange assay (Margarit et al., Cell, 112:655-695, 2003). Other assays far measuring Ras and Erk activation are known in the art.

Any combination of the above methods can also be used. The above methods can be used to evaluate any genetic locus, e.g., in a method for analyzing genetic information from particular groups of individuals or in a method for analyzing a mutation associated with Noonan syndrome of a neoplastic disorder, e.g., the SOS1 locus, or the PTPN11 locus. The methods can be performed in conjunction with methods to detect mutations in other genes associated with cancer or NS. See. e.g., U.S. Pat. Pub. No. 20030125298, describing methods and compositions for detecting PTPN11 mutations and diagnosing NS (herein incorporated by reference in its entirety).

### Evaluating Noonan Syndrome and Other Disorders

Noonan syndrome is assigned to MIM 163950 in the Online Mendelian inheritance in Man (OMIM) database at the World Wide Web Address www.ncbi.nlm.nih.gov/Omim, A variety of criteria, can be used to evaluate whether a subject has Noonan syndrome, or to evaluate whether a subject is at risk for developing Noonan syndrome. These criteria can be evaluated in conjunction with efforts to determine whether the subject carries a mutation in a SOS1 gene. The criteria include biochemical, physiological, and cognitive criteria, as well as genetic evaluation (e.g., evaluation of other loci associated with Noonan syndrome). Phenotypic characteristics of NS include: cardiac defects (e.g., hypertrophic cardiomyopathy, pulmonic stenosis, atrial septal defect, and aortic coarctation), dysmorphic facial features (e.g., broad forehead, hypertelorism, down-slanting palpebral fissures, highly arched palate, low set and posteriorly rotated ears), proportionate short stature, pectus deformity, cryptorchidism, developmental delay, genitourinary malformations, bleeding disorders, lymphatic dysplasia, and growth failure. See Tartagha and Gelb, Noonan Syndrome and Related Disorders: Genetics and Pathogenesis, Annu Rev Genomics Hum Genet 6, 45-68 (2.005). Information about these features and other features known to be associated with NS can be used in various methods described herein.

Determining whether an individual carries a SOS1 mutation can facilitate in distinguishing NS from related disorders, such as cardio-facial-cutaneous syndrome (CFC, MIM 115150 in the OMIM database), or Costello syndrome (MIM 218040). In certain embodiments, the presence of a SOS1 mutation indicates that a subject has NS rather than one of these related disorders.

Subjects being diagnosed for NS or a neoplastic disorder may exhibit biochemical abnormalities that result from the pathology of the disease. Techniques to detect biochemical abnormalities in a sample from a subject include cellular, immunological, and other biological methods known in the art. For general guidance, see, e.g., techniques described in Sambrook & Russell, Molecular Cloning: A Laboratory Manual, 3^{rd} Edition, Cold Spring Harbor Laboratory, N.Y. (2001), Ausubel at., Current Protocols in Molecular Biology (Greene Publishing Associates and Wiley Interscience, N.Y. (1989), (Harlow, H. and Lane, D. (1988) Antibodies: A Laboratory Manual, Cold Spring Harbor Laboratory Press, Cold Spring Harbor, N.Y.), and updated editions thereof.

For example, antibodies, other immunoglobulins, and other specific binding ligands can be used to detect a biomolecule, e.g., a protein or other antigen associated with NS or a neoplastic disorder. For example, one or more specific antibodies can be used to probe a sample. Various formats are possible, e.g., ELISAs, fluorescence-based assays. Western blots, and protein arrays. Methods of producing polypeptide arrays are described in the art, e.g., in De Wildt et al. (2000). Nature Biotech. 18, 989-994: Lucking et al. (1999). Anal. Biochem, 270, 103-111; Ge, H. (2000). Nucleic Acids Res. 28, e3, I-VII; MacBeath, G., and Schreiber, S. L. (2000). Science 289, 1760-1763; and WO 99/51773A1.

Proteins can also be analyzed using mass spectroscopy, chromatography, electrophoresis, enzyme interaction or using probes that detect post-translational modification (e.g., a phosphorylation, ubiquitination, glycosylation, methylation, or acetylation).

Nucleic acid expression can be detected in cells from a subject, e.g., removed by surgery, extraction, post-mortem or other sampling (e.g., blood, CSF, amniotic fluid). Expression of one or more genes can be evaluated, e.g., by hybridization based techniques, e.g., Northern analysis, RT-PCR, SAGE, and nucleic acid arrays. Nucleic acid arrays are useful for profiling multiple mRNA species in a sample. A nucleic acid array can be generated by various methods, e.g., by photolithographic methods (sec, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No 5,288,514), and head-based techniques (e.g., as described in PCT US/93/04145)

The array typically includes oligonucleotide probes capable of specifically hybridizing to different polymorphic variants. A nucleic acid of interest, e.g., a nucleic acid encompassing a mutation site, (which is typically amplified) is hybridized with the array and scanned. Hybridization and scanning are generally carried out according to standard methods. See, e.g., Published PCT Application Nos. WO 92/10092 and WO 95/11995, and U.S. Pat. No. 5,424,186. After hybridization and washing, the array is scanned to determine the position on the array to which the nucleic acid hybridizes. The hybridization data obtained from the scan is typically in the form of fluorescence intensities as a function of location on the array.

Arrays can include multiple detection blocks (i.e., multiple groups of probes designed for detection of particular mutations). Such arrays can be used to analyze multiple different mutations in multiple genes. Detection blocks may be grouped within a single array or in multiple, separate arrays so that varying conditions (e.g., conditions optimized for particular mutations) may be used during the hybridization. For example, it may be desirable to provide for the detection of those polymorphisms that fall within G-C rich stretches of a genomic sequence, separately from those falling in A-T rich segments. Additional description of use of oligonucleotide arrays for detection of polymorphisms can be found, for example, in U.S. Pat. Nos. 5,858,659 an 5,837,832. In addition to oligonucleotide arrays, cDNA arrays may be used similarly in certain embodiments of the invention.

The methods described herein can include providing an array; contacting the array with a sample, e.g., a portion of genomic DNA that includes at least a portion of human SOS1 gene, and, e.g., another genomic region associated with NS (e.g., a genomic region which includes at least a portion of a PTPN11 gene), and detecting binding of a nucleic acid from the sample to the array.

Metabolites that are associated with NS or a neoplastic disorder can be detected by a variety of means, including enzyme-coupled assays, using labeled precursors, and nuclear magnetic resonance (NMR). For example, NMR can be used to determine the relative concentrations of phosphate-based compounds in a sample, e.g., creatine levels. Other metabolic parameters such as redox state, ion concentration (e.g., Ca²⁺) (e.g., using ion-sensitive dyes), and membrane potential can also be detected (e.g., using patch-clamp technology).

Information about an NS—or neoplastic disorder-associated marker can be recorded and/or stored in a computer-readable format. Typically the information is linked to a reference about the subject and also is associated (directly or indirectly) with information about the identity of one or more nucleotides in the subject's SOS1 genes.

### Data Analyses

Certain aspects of the invention can be implemented in digital electronic circuitry, or in computer hardware, firmware, software, or in combinations thereof. Methods of the invention can be implemented using a computer program product tangibly embodied in a machine-readable storage device for execution by a programmable processor; and method actions can be performed by a programmable processor executing a program of instructions to perform functions of the invention by operating on input data and generating output. For example, the invention can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object oriented programming language, or in assembly or machine language if desired; and in any ease, the language can be a compiled or interpreted language. Suitable processors include, by way of example, both general and special purpose microprocessors. A processor can receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks such as, internal hard disks and removable disks; magneto-optical disks; and CD_ROM disks. Any of the foregoing can be supplemented by, or incorporated in, ASICs (application-specific integrated circuits).

In one implementation, information about a set of potential reference sequences and/or reference subjects (e.g., NS-affected subjects, or subjects who are not affected with NS) is stored on a server. A user can send information about case groups to the server, e.g., from a remote computer that communicates with the server using a network, e.g., the Internet. The case groups can be, e.g., individuals at risk for NS. The server can compare the information about the test sequences and/or test subjects and select a subset of members from the potential controls, e.g., to minimize a distance measure that is a function of the case groups and the selected subset. The server can return information about the subset (e.g., identifiers or other data) to the user or can return an evaluation that compares a feature of the case group to the members of the selected subset (e.g., a statistical score that evaluates probability of association with the case group relative to the selected subset). Accordingly, the server can include a electronic interface for receiving information from a user or from an apparatus that provides information about a biological property and software configured to execute identify a subset of data objects using a comparison described herein.

In another implementation, information about a subject's SOS1 locus (e.g., information about one or both SOS1 alleles) is stored on a server. A user can send information about the subject (e.g., a patient, a relative of a patient, a sample of gametes (e.g., sperm or oocytes), fetal cells, or a candidate for a treatment) to the server, e.g., from a remote computer that communicates with the server using a network, e.g., the Internet. The server can compare the information about the subject, e.g., to reference information to produce an indication as to the individual propensity for NS and/or cancer. For example, the reference information can be information derived from a reference individual, a particular sequence, or a population of sequences. The indication can be, for example, qualitative or quantitative. An exemplary qualitative indication includes a binary output or a descriptive output (e.g., text or other symbols indicating degree of propensity for NS). An exemplary qualitative indication includes a statistical measure of the probability of developing NS, a score, a percentage, or a parameter for a risk evaluation (e.g., a parameter that can be used in a financial evaluation).

It is also possible for the server to return the indication or information about related subjects (e.g., family members or subjects with similar SOS1 loci), e.g., to the user. For example, the server can build a family tree used on a set of related subject. Each individual can be, e.g., assigned a statistical score that evaluates probability of NS as a function of an NS-associated gene locus, e.g., the SOS1 locus, and/or other factors. Accordingly, the server can include an electronic interface for receiving information from a user or from an apparatus that provides information about an NS-associated gene locus.

In one method, information about the subject's SOS1 locus, e.g., the result of evaluating a mutation in the SOS1 gene, as described herein, is provided (e.g., communicated, e.g., electronically communicated) to a third party, e,g., a hospital, clinic, a government entity, reimbursing party or insurance company (e.g., a life insurance company). For example, choice of medical procedure, payment for a medical procedure, payment by a reimbursing party, or cost for a service or insurance can be function of the information.

In one embodiment, a premium for insurance (e.g., life or medical) is evaluated as a function of information about one or more NS- or cancer-associated mutations, e.g., a mutation described herein, e.g., a SOS1 mutation. For example, premiums can be increased (e.g., by a certain percentage) if a first mutation is present in the candidate insured, or decreased if a second mutation is present. Premiums can also be scaled depending on heterozygosity or homozygosity. For example, premiums can be assessed to distribute risk, e.g., commensurate with the allele distribution for the particular mutation. In another example, premiums are assessed as a function of actuarial data that is obtained from individuals with one or more NS-associated mutations.

Genetic information about one or more NS-associated mutations, e.g., a mutation described herein, e.g., a SOS1 mutation, can be used e.g., in an underwriting process for life insurance. The information can be incorporated into a profile about a subject. Other information in the profile can include, for example, date of birth, gender, marital status, banking information, credit information, children and so forth. An insurance policy can be recommended as a function of the genetic information along with one or more other items of information in the profile. An insurance premium or risk assessment can also be evaluated as function of the genetic information. In one implementation, points are assigned for presence or absence of a particular allele. The total points for NS-associated mutations and other risk parameters are summed. A premium is calculated as a function of the points, and optionally one or more other parameters.

In one embodiment, information about an NS-associated polymorphism, e.g., a mutation described herein is analyzed by a function that determines whether to authorize or transfer of funds to pay for a service or treatment provided to a subject. For example, an allele that is not associated with NS can trigger an outcome that indicates or causes a refusal to pay for a service or treatment provided to a subject. For example, an entity, e.g., a hospital, care giver, government entity, or an insurance company or other entity which pays for, or reimburses medical expenses, can use the outcome of a method described herein to determine whether a party, e.g., a party other than the subject patient, will pay for services or treatment provided to the patient. For example, a first entity, e.g., an insurance company, can use the outcome of a method described herein to determine whether to provide financial payment to, or on behalf of, a patient, e.g., whether to reimburse a third party, e.g., a vendor of goods or services, a hospital, physician, or other care-giver, for a service or treatment provided to a patient. For example, a first entity, e.g., an insurance company, can use the outcome of a method described herein to determine whether to continue, discontinue, enroll an individual in an insurance plan or program, e,g., a health insurance or life insurance plan or program.

### Pharmacogenomics

Both prophylactic and therapeutic methods of treatment may be specifically tailored or modified, based on knowledge obtained from a pharmarogenomics analysis. In particular, a subject can be treated based on the presence or absence of a genetic mutation associated with NS or a neoplastic disorder, e.g., a SOS1 mutation. Pharmacogenomics allows a clinician or physician to target prophylactic or therapeutic treatments to patients who will most benefit from the treatment and to avoid the treatment of patients who will experience toxic or other undesirable drug-related side effects. In particular, a diet or drug that affects NS or cancer can be prescribed as a function of the subject's SOS1 locus. For example, if an individual's SOS1 gene includes a mutation, the individual can be indicated for a prophylactic treatment for a drug that alleviates a symptom of the cancer. In another example, the individual is placed in a monitoring program, e.g., to closely monitor for physical manifestations of cancer or NS onset.

### Screening Assays

The invention includes methods of screening for compounds that modulate SOS1 activity, particularly compounds that modulate the activity of hypermorphic SOS1 mutants and/or compounds that decrease SOS1 activity. Such compounds include a compound which directly interacts with SOS1 and compounds which alter SOS1 protein or RNA expression. Such compounds can be identified as candidates for the prevention or treatment of NS and neoplastic disorders. Screening methods can be employed to identify compounds that specifically modulate activity of mutant SOS1 polypeptides, wild type SOS1 polypeptides, or both. Therefore, in certain embodiments, a wild type SOS1 polypeptide can be used in place of a mtSOS1 polypeptide, or vice versa.

One method can include providing a compound which interacts with SOS1, or a mutant thereof, and evaluating the effect of the compound on a biochemical, cellular, or organismal phenotype associated with NS or cancer, e.g., as described herein. Another method can include screening for compounds using a method that includes evaluating the compounds for modulation of SOS1 activity and evaluating the effect of the compound on a biochemical, cellular, or organismal phenotype associated with NS. The evaluations can be performed in either order. For example, a library of compounds can be vetted using the first criterion (modulation of SOS1 activity) to provide a smaller set of compounds, and then evaluating compounds from the smaller set for an effect on an NS or neoplastic phenotype. The vetting can also be done in the opposite order.

Compounds which interact with SOS1 can be identified, e.g., by in vitro or in vivo assays. Exemplary in vitro assays for SOS1 activity include cell free assays, e.g., assays in which an isolated SOS1 polypeptide (including a polypeptide that includes a fragment of at least 100 amino acids of SOS1, e.g., a fragment described herein) is contacted with a test compound.

When both the assay for screening a compound for the ability to interact with SOS1 and the assay for determining effect on SOS1 are performed in vivo, e.g., in cell based assays, the assays can be performed in the same or different cells. For example, one or both of the assays can be performed in tissue culture (e.g., 293T cells) or in an organism (e.g., a mammal e.g., a human, or a mouse).

Described below are exemplary methods for identifying compounds that interact with SOS1, or a mutant thereof, and can modulate SOS1 activity or expression. Preferably, compounds can be identified which interact with, e,g., bind to, SOS1 or mtSOS1 and decrease a SOS1-mediated activity, such as activation of a Ras/Erk signaling pathway.

A variety of techniques may be utilized to modulate the expression, synthesis, or activity of such target genes and/or proteins. Such molecules may include, but are not limited to small organic molecules, peptides, antibodies, nucleic acids, antisense nucleic acids, RNAi, ribozyme molecules, triple helix molecules, and the like.

The following assays provide methods (also referred to herein as “evaluating a compound” or “screening a compound”) for identifying modulators, i.e., candidate or test compounds (e.g., peptides, peptidomimetics, small molecules or other drugs) which interact with and/or modulate SOS1 activity, e.g., have a stimulatory or inhibitory effect on, for example, SOS1 expression and/or activity, or have a stimulatory or inhibitory effect on, for example, the expression or activity of a SOS1 substrate. Such compounds can be agonists or antagonists of SOS1, In preferred embodiments, the screening assays described herein are used to identify candidates which function as SOS1 antagonists. As described herein, such a SOS1 antagonist can decrease Ras signaling of a cell, which has practical utility, e.g., in NS prevention or treatment and/or cancer prevention or treatment. Some of these assays may be performed in animals, e.g., mammals, in organs, in cells. Others may be performed in animals, e.g., mammals, in organs, in cells, in cell extracts, e.g., purified or unpurified nuclear extracts, intracellular extracts, in purified preparations, in cell-free systems, in cell fractions enriched for certain components, e.g., organelles or compounds, or in other systems known in the art. Given the teachings herein and the state of the art, a person of ordinary skill in the art would be able to choose an appropriate system and assay for practicing the methods of the present invention.

A “compound” or “test compound” can be any chemical compound, for example, a macromolecule (e.g., a polypeptide, a protein complex, or a nucleic acid) or a small molecule (e.g., an amino acid, a nucleotide, an organic or inorganic compound). The test compound can have a formula weight of less than about 10,000 grams per mole, less than 5,000 grams per mole, less than 1,000 grams per mole, or less than about 500 grams per mole. The test compound can be naturally occurring (e.g., a herb or a nature product), synthetic, or both. Examples of macromolecules are proteins, protein complexes, and glycoproteins, nucleic acids, e.g., DNA, RNA (e.g., double stranded RNA or RNAi) and PNA (peptide nucleic acid). Examples of small molecules are peptides, peptidemimetics (e.g., peptoids), amino acids, amino acid analogs, polynucleotides, polynucleotide analogs, nucleotides, nucleotide analogs, organic or inorganic compounds e.g., heteroorganic or organometallic compounds. One exemplary type of protein compound is an antibody or a modified scaffold domain protein. A test compound can be the only substance assayed by the method described herein. Alternatively, a collection of test compounds can be assayed either consecutively or concurrently by the methods described herein.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display as desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Intl. J. Pept. Prot. Res, 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like). Additional examples of methods for the synthesis of molecular libraries can be found in the art, for example in: DeWitt et al. (1993) Proc. Natl. Acad. Sci. USA, 90:6909; Erb et al. (1994) Proc. Natl. Acad. Sci. USA 91:11422; Zuckerman et al. (1994), J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Some exemplary libraries are used to generate variants from a particular lead compound. One method includes generating a combinatorial library in which one or more functional groups of the lead compound are varied, e.g., by derivatization. Thus, the combinatorial library can include a class of compounds which have a common structural feature (e.g., framework).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripes, Inc., St. Louis, Mo., ChemStar, Ltd, Moscow, RU, 3D Pharmaceuticals, Exton, Pa., Martek Biosciences, Columbia, Md., etc.).

The test compounds of the present invention can also be obtained from: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological libraries include libraries of nucleic acids and libraries of proteins. Some nucleic acid libraries encode a diverse set of proteins (e.g., natural and artificial proteins; others provide, for example, functional RNA and DNA molecules such as nucleic acid aptamers or ribozymes. A peptoid library can be made to include structures similar to a peptide library. (See also Lam (1997) Anticancer Drug Des. 12:145). A library of proteins may be produced by an expression library or a display library (e.g., a phage display library).

Libraries of compounds may be presented in solution (e.g. Houghten (1992) Biotechniques 13:412-421), or on beads (Lam (1991) Nature 354:82-84), chips (Fodor (9931) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In Vitro Assays

Exemplary in vitro assays include assays for a binding interaction or a catalytic activity, e.g., Ras-guanine nucleotide exchange. In some embodiments, interaction with, e.g., binding of, SOS1 can be assayed in vitro. The reaction mixture can include a SOS1 binding partner, e.g., Ras, Grb2, and compounds can be screened, e.g., in an in vitro assay, to evaluate the ability of a test compound to modulate interaction between SOS1 and a SOS1 binding partner. This type of assay can be accomplished, for example, by coupling one of the components, with a radioisotope or enzymatic label such that binding of the labeled component to the other can be determined by detecting the labeled compound in a complex. A component can be labeled with ¹²⁵I, ³⁵S, ¹⁴C, or ³H, either directly or indirectly, and the radioisotope detected by direct counting of radioemmission or by scintillation counting. Alternatively, a component can be enzymatically labeled with, for example, horseradish peroxidase, alkaline phosphatase, or luciferase, and the enzymatic label detected by determination of conversion of an appropriate substrate to product. Competition assays can also be used to evaluate a physical interaction between a test compound and a target.

Cell-free assays involve preparing a reaction mixture of the target protein (e.g., SOS1) and the test compound under conditions and for a time sufficient to allow the two components to interact and bind, thus forming a complex that can be removed and/or detected.

The interaction between two molecules can also be detected, e.g., using a fluorescence assay in which at least one molecule is fluorescently labeled. One example of such an assay includes fluorescence energy transfer (FET or FRET for fluorescence resonance energy transfer) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A FRET binding event can be conveniently measured through standard fluorometric detection means well known in the art (e.g., using a fluorimeter).

Another example of a fluorescence assay is fluorescence polarization (FP). For FP, only one component needs to be labeled. A binding interaction is detected by a change in molecular size of the labeled component. The size change alters the tumbling rate of the component in solution and is detected as a change in FP. See, e.g., Nasir et al. (1999) Comb Chem HTS 2:177-190; Jameson et al. (1995) Methods Enzymol 246:283; Seethala et al., (1998) Anal Biochem. 255:257. Fluorescence polarization can be monitored in multiwell plates, e.g., using the Tecan Polarion™ reader. See, e.g., Parker et al. (2000) Journal of Biomolecular Screening 5:77-88; and Shoeman, et al., (1999) 38, 16802-16809.

In another embodiment, determining the ability of the SOS1 protein to bind to a target molecule can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SFR)), resulting in a detectable signal which can be used as an indication of real-time reactions between biological molecules.

In one embodiment, SOS1 is anchored onto a solid phase. The SOS1/test compound complexes anchored on the solid phase can be detected at the end of the reaction, e.g., the binding reaction. For example, SOS1 can be anchored onto a solid surface, and the test compound, (which is not anchored), can be labeled, either directly or indirectly, with detectable labels discussed herein.

It may be desirable to immobilize either the SOS1 or an anti-SOS1 antibody to facilitate separation of complexed from uncomplexed forms of one or both of the proteins, as well as to accommodate automation of the assay. Binding of a test compound to a SOS1 protein, or interaction of a SOS1 protein with a second component in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/SOS1 fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or SOS1 protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of SOS1 binding or activity determined using standard techniques.

Other techniques for immobilizing either a SOS1 protein or a target molecule on matrices include using conjugation of biotin and streptavidin. Biotinylated SOS1 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface, e,g., using a labeled antibody specific for the immobilized component (the antibody, in turn, can be directly labeled or indirectly labeled with, e.g., at labeled anti-Ig antibody).

In one embodiment, this assay is performed utilizing antibodies reactive with a SOS1 protein or target molecules but which do not interfere with binding of the SOS1 protein to its target molecule. Such antibodies can be derivatized to the wells of the plate, and unbound target or the SOS1 protein trapped in the wells by antibody conjugation. Methods for detecting such complexes, in addition to those described above for the GST-immobilized complexes, include immunodetection of complexes using antibodies reactive with the SOS1 protein or target molecule, as well as enzyme-linked assays which rely on detecting an enzymatic activity associated with the SOS1 protein or target molecule.

Alternatively, cell free assays can be conducted in it liquid phase. In such an assay, the reaction products are separated from unreacted components, by any of a number of standard techniques, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999. J. Wiley: New York.); and immunoprecipitation (see, for example, Ausubel, F. et al., eds. (1999) Current Protocols in Molecular Biology, J. Wiley: New York). Such resins and chromatographic techniques are known to one skilled in the art. (see, e.g., Heegaard, N. H., (1998) J. Mol Recognit 11:141-8; Hage, and Tweed, S. A. (1997) J. Chromatogr B Biomed Sci. Appl. 699:499-525). Further, fluorescence energy transfer may also be conveniently utilized, as described herein, to detect binding without further purification of the complex from solution.

In a preferred embodiment, the assay includes contacting the SOS1 protein or biologically active portion thereof with a known compound which binds a SOS1 to form an assay mixture, contacting the assay mixture with a test compound, and determining the ability of the test compound to interact with a SOS1 protein, wherein determining the ability of the test compound to interact with the SOS1 protein includes determining the ability of the test compound to preferentially bind to the SOS1 or biologically active portion thereof, or to modulate the activity of a target molecule, as compared to the known compound.

The target products of the invention can, in vivo, interact with one or more cellular or extracellulas macromolecules, such as proteins. For the purposes of this discussion, such cellular and extracellular macromolecules are referred to herein as “binding partners.” Compounds that disrupt such interactions can be useful in regulating the activity of the target product. Such compounds can include, but are not limited to molecules such as antibodies, peptides, and small molecules. The preferred targets/products for use in this embodiment include the SOS1 binding partners.

To identify compounds that interfere with the interaction between the target product and its binding partner(s), a reaction mixture containing the target product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target and its cellular or extracellular binding partner. Control reaction mixtures are incubated without the test compound or with a placebo. The formation of any complexes between the target product and the cellular or extracellular binding partner is then detected. The formation of a complex in the control reaction, but not in the reaction mixture containing the test compound, indicates that the compound interferes with the interaction of the target product and the interactive binding partner. Additionally, complex formation within reaction mixtures containing the test compound and normal target product can also be compared to complex formation within reaction mixtures containing the test compound and mutant target product. This comparison can be important in those cases wherein it is desirable to identify compounds that disrupt interactions of mutant but not normal target products.

These assays can be conducted in a heterogeneous or homogeneous format. Heterogeneous assays involve anchoring either the target product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target product or the partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target product and the interactive cellular or extracellular binding partner product is prepared in that either the target products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target product-binding partner interaction can be identified.

In yet another aspect, the SOS1 proteins can be used as “bait proteins” in a two-hybrid assay or three-hybrid assay (see, e.g., U.S. Pat. No. 5,283,17; Zervos et al. (1993) Cell 72:223-232; Madura et al. (1993) J. Biol. Chem. 268:12046-12054; Bartel et al. (1993) Biotechniques 14920-924; Iwabuchi et al. (1993) Oncogene 8:1693-1696; and Brent WO94/10304 to identify other proteins, which bind to or interact with SOS1 (“SOS1-binding proteins”) and are involved in SOS1 activity. Such SOS1 binding partners can be activators or inhibitors of signals by the SOS1 proteins.

In another embodiment, modulators of SOS1 gene expression are identified. For example, a cell or cell free mixture is contacted with a candidate compound and the expression of the SOS1 mRNA or protein evaluated relative to the level of expression of SOS1 mRNA or protein in the absence of the candidate compound. When expression of the SOS1 mRNA or protein is less (statistically significantly less) in the presence of the candidate compound than in its absence, the candidate compound is identified as an inhibitor of the SOS1 mRNA or protein expression. The level of the SOS1 mRNA or protein expression can be determined by methods for detecting SOS1 mRNA or protein, e.g., using probes or antibodies, e.g., labeled, probes or antibodies.

Cell-Based Assays

Cell-based assays can be used to evaluate SOS1 activity in a cell and also as a cell-based method to evaluate a compound for an effect on NS or a neoplastic disorder. Useful assays include assays in which a Ras-MAPK pathway is measured, described elsewhere herein.

An exemplary cell based assay can include contacting a cell expressing SOS1 with a test compound and determining the ability of the test compound to modulate (e.g. stimulate or inhibit) an activity of SOS1, and/or determine the ability of the test compound to modulate SOS1 expression, e.g., by detecting SOS1 nucleic acids (e.g., mRNA or cDNA) or proteins in the cell. A preferred activity is the stimulation of Ras or Erk activation.

In some embodiments, the cells can be recombinant or non-recombinant cells which express a SOS1 binding partner or substrate (natural or artificial). Preferred systems include mammalian or yeast cells that express SOS1. In utilizing such systems, cells are exposed to compounds suspected of increasing SOS1 expression and/or SOS1 activity. After exposure, the cells are assayed, for example, for expression of the SOS1 gene or activity of the SOS1 protein.

A cell used in the methods of the invention can be from a stable cell line or a primary culture obtained from an organism, e.g., a organism treated with the test compound.

In addition to cell-based and in vitro assay systems, non-human organisms, e.g., transgenic non-human organisms, can also be used. A transgenic organism is one in which a heterologous DNA sequence is chromosomally integrated into the germ cells of the animal. A transgenic organism will also have the transgene integrated into the chromosomes of its somatic cells. Organisms of any species, including, but not limited to yeast, worms, flies, fish, reptiles, birds, mammals (e.g., mice, rats, rabbits, guinea pigs, pigs, micro-pigs, and goats), and non-human primates (e.g., baboons, monkeys, chimpanzees) may be used in the methods of the invention. Transgenic mouse models (e.g., mouse models expressing a mutant human SOS1 polypeptide described herein or a marine ortholog of the mutant human SOS1 polypeptide) can be used in the methods of the invention.

### Pharmaceutical Compositions

The invention includes methods of modulating SOS1 activity, e.g., to treat or prevent NS or a neoplastic disorder. The method can include administering to a cell or an organism a compound that interacts with SOS1 and effects SOS1 activity. For example, the compound can be a SOS1 antagonist. In some embodiments, the compound specifically interacts with a mutant SOS1 polypeptide.

The compound can be administered to human or a human cell. The compound can also be administered to other types of cells and organisms, e.g., for evaluation in in vitro or in animal models of NS or cancer. For example, the cell to which the compound is administered can be an invertebrate cell, e.g., a worm cell or a fly cell, or a vertebrate cell, e.g., a fish cell (e.g., zebrafish cell), or a mammalian cell (e.g., mouse). Similarly, the organism to which the compound is administered can be an invertebrate, e.g., a worm or a fly, or a vertebrate, e.g., a fish (e.g., zebrafish), an amphibian, or a mammal (e.g., rodent).

The compound can be a small organic compound, an antibody, a polypeptide, or a nucleic acid molecule.

Antibodies that are both specific for a target gene protein and that interfere with its activity may be used to inhibit function of a target protein, e.g., a negative regulator of SOS1 (e.g., the SOS1 gene or protein). Such antibodies may be generated using standard techniques, against the proteins themselves or against peptides corresponding to portions of the proteins. Such antibodies include but are not limited to polyclonal, monoclonal, Fab fragments, single chain antibodies, chimeric antibodies, humanized antibodies and the like. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art.

The SOS1 antagonist can also be a siRNA, anti-sense RNA, or a ribozyme which can decrease the expression of the SOS1 polypeptide (e.g., by inhibiting expression of SOS1). Double-stranded inhibitory RNA is particularly useful as it can be used to selectively reduce the expression of one allele of a gene and not the other, thereby achieving an approximate 50% reduction in the expression of a SOS1 polypeptide, See Garrus et al. (2001), Cell 107(1):55-65. Thus, in some aspects, a cell or subject can be treated with a compound that modulates the expression of a gene, e.g., a nucleic acid which modulates, e.g., decreases, expression of a SOS1 polypeptide. Such approaches include oligonucleotide-based therapies such as RNA interference, antisense, ribozymes, and triple helices.

dsRNA can be delivered to cells or to an organism to antagonize SOS1. Endogenous components of the cell or organism trigger RNA interference (RNAi) which silences expression of genes that include the target sequence, dsRNA can be produced by transcribing a cassette (in vitro or in vivo) in both directions, for example, by including a T7 promoter on either side of the cassette. The insert in the cassette is selected so that it includes a sequence complementary to a nucleic acid encoding SOS1. The sequence need not be full length, for example, an exon, or at least 50 nucleotides. The sequence can be from the 5′ half of the transcript, e.g., within 1000, 600, 400, or 300 nucleotides of the ATG. See also, the HiScribe™ RNA: Transcription Kit (New England Biolabs, Mass.) and Fire, A. (1999) Trends Genet. 15, 358-363. dsRNA can be digested into smaller fragments. See, e.g., US Patent Pub. Nos. 2002-0086356 and 2003-0084471. In one embodiment, an siRNA is used, siRNAs are small double stranded RNAs (dsRNAs) that optionally include overhangs. For example, the duplex region is about 18 to 25 nucleotides in length, e.g., about 19, 20, 21, 22, 23, or 24 nucleotides in length. Typically the siRNA sequences are exactly complementary to the target mRNA. siRNA includes short hairpin RNA (shRNA), which is an RNA molecule comprising, at least two complementary portions hybridized or capable of hybridizing to form a double-stranded (duplex) structure sufficiently long to mediate RNAi. MicroRNAs are also contemplated (see, e.g., Ruvkun, G., Science, 294, 797-799, 2001; Zeng, Y., et al. Molecular Cell, 9, 1-20, 2002).

Oligonucleotides may be designed to reduce or inhibit mutant target gene expression and/or activity. Techniques for the production and use of such molecules are well known to those of ordinary skill in the art. Antisense RNA and DNA molecules act to directly block the translation of mRNA by hybridizing to targeted mRNA and preventing protein translation. With respect to antisense DNA, oligodeoxyribonucleotides derived from the translation initiation site, e.g., between the −10 and +10 regions of the target gene nucleotide sequence of interest, are preferred. Antisense oligonucleotides are preferably 10 to 50 nucleotides in length, and more preferably 15 to 30 nucleotides in length. An antisense compound is an antisense molecule corresponding to the entire mRNA of the target gene or fragments thereof.

Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. The composition of ribozyme molecules includes one or more sequences complementary to the target gene mRNA, and includes the well known catalytic sequence responsible for mRNA cleavage disclosed, for example, in U.S. Pat. No. 5,093,246. Within the scope of this disclosure are engineered hammerhead motif ribozyme molecules that specifically and efficiently catalyze endonucleolytic cleavage of RNA sequences encoding target gene proteins. Specific ribozyme cleavage sites within any potential RNA target are initially identified by scanning the molecule of interest for ribozyme cleavage sites that include the sequences GUA, GUU, and GUC. One identified, short RNA sequences of between 15 and 20 ribonucleotides corresponding to the region of the target gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays.

The antisense, ribozyme, and/or triple helix molecules described herein may reduce or inhibit the transcription (triple helix) and/or translation (antisense, ribozyme) of mRNA produced by both normal and mutant target gene alleles.

Antisense RNA and DNA, RNAs that mediate RNAi, ribozyme, and triple helix molecules may be prepared by any method known in the art for the synthesis of DNA and RNA molecules. These include techniques for chemically synthesizing oligodeoxyribonucleotides and oligoribonucleotides, for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. Various well-known modifications to the DNA molecules may be introduced as a means of increasing intracellular stability and half-life. Possible modifications include but are not limited to the addition of flanking sequences of ribonucleotides or deoxyribonucleotides of the 5′ and/or 3′ ends of the molecule or the use of phosphorothioate or 2′ O-methyl rather than phosphodiesterase linkages within the oligodeoxyribonucleotide backbone.

Delivery of nucleic acids can be achieved using a recombinant expression vector such as a chimeric virus or a colloidal dispersion system or by injection. Useful virus vectors include adenovirus, herpes virus, vaccinia, and/or RNA virus such as a retrovirus. The retrovirus can be a derivative of a routine or avian retrovirus such as Moloney murine leukemia virus or Rous sarcoma virus. All of these vectors can transfer or incorporate a gene for a selectable marker so that transduced cells can be identified and generated. The specific nucleotide sequences that can be inserted into the retroviral genome to allow target specific delivery of the retroviral vector containing an antisense oligonucleotide can be determined by one of skill in the art.

Another delivery system for polynucleotides is a colloidal dispersion system. Colloidal dispersion systems include macromolecular complexes, nanocapsules, microspheres, beads, and lipid-based systems including oil-in-water emulsions, micelles, mixed micelles and liposomes. A preferred colloidal delivery system is a liposome, an artificial membrane vesicle useful as in vivo or in vitro delivery vehicles. The composition of a liposome is usually a combination of phospholipids, usually in combination with steroids, particularly cholesterol.

The identified compounds that modulate (e.g., inhibit) SOS1 activity, e.g., SOS1 gene expression, synthesis and/or activity (or inhibit expression of a target gene product that activates SOS1) can be administered to a patient at therapeutically effective doses to treat or ameliorate or delay one or more of the symptoms of NS or cancer. A therapeutically effective dose refers to that amount of the compound sufficient to result in amelioration or delay of one or more of the symptoms of NS or cancer.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Pharmaceutical compositions may be formulated in conventional manner using one or more physiologically acceptable carriers or excipients. Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups, or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring, and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing as valve to deliver a metered amount. Capsules and cartridges of e.g. gelatin far use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, and/or dispersing agents. Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compounds identified by the methods described herein can be used in the treatment of conditions associated with NS or a neoplastic disorder. The compounds can be administered alone or as mixtures with conventional excipients, such as pharmaceutically, or physiologically, acceptable organic, or inorganic carrier substances such as water, salt solutions (e.g., Ringer's solution), alcohols, oils and gelatins. Such preparations can be sterilized and, if desired, mixed with lubricants, preservatives, stabilizers, wetting agents, emulsifiers, salts for influencing osmotic pressure, buffers, coloring, and/or aromatic substances and the like.

Therapeutic Uses

The invention includes methods for treating or preventing a condition in which SOS (e.g., SOS1) is implicated, e.g., NS or cancer, in a subject. The method includes administering a SOS antagonist. For example, the SOS antagonist can be one or more of: a SOS1 nucleic acid, RNAi (e.g., RNAi targeted to a molecule that inhibits SOS1), and other compounds identified by a method described herein, e.g., compounds that inhibit SOS1. The invention also includes methods for treating or preventing such conditions with agents that target genes or gene products implicated in SOS/Ras signaling, such as antagonists of Ras, Raf, MEK, Erk, Rsk, PI3 Kinase, Akt, Tor, Pak, and farnesyltransferase inhibitors.

“Subject,” as used herein, refers to human and non-human animals. The term “non-human animals” of the invention includes all vertebrates, e.g., mammals, such as non-human primates (particularly higher primates), sheep, dog, rodent (e.g., mouse or rat), guinea pig, goat, pig, cat, rabbits, cow, and non-mammals, such as chickens, amphibians, reptiles, etc. In a preferred embodiment, the subject is a human, e.g., an NS or cancer patient. In another embodiment, the subject is an experimental animal or animal suitable as a disease model.

In one embodiment, the method includes administering a SOS1 antagonist in combination with one or more additional therapeutic agent or agents, e.g., a therapeutic agent or agents for treating NS or cancer.

### Examples

### Example 1

We studied a cohort of 91 probands with a confirmed diagnosis of NS (see Methods), of whom 34 (37%) had a missense mutation in PTPN11. All exons and parts of flanking intronic sequences for the CSK, PTPN6, PAG, MRAS, and SOS2 genes in the remaining 57 probands were sequenced, and no coding sequence variants were found. Also, as expected from its more distal position in the RAS/ERK pathway and specific association with CFC, no BRAF mutations were identified. However, a substantial number of mutations in SOS1, which encodes a major RAS-GEF, were discovered.

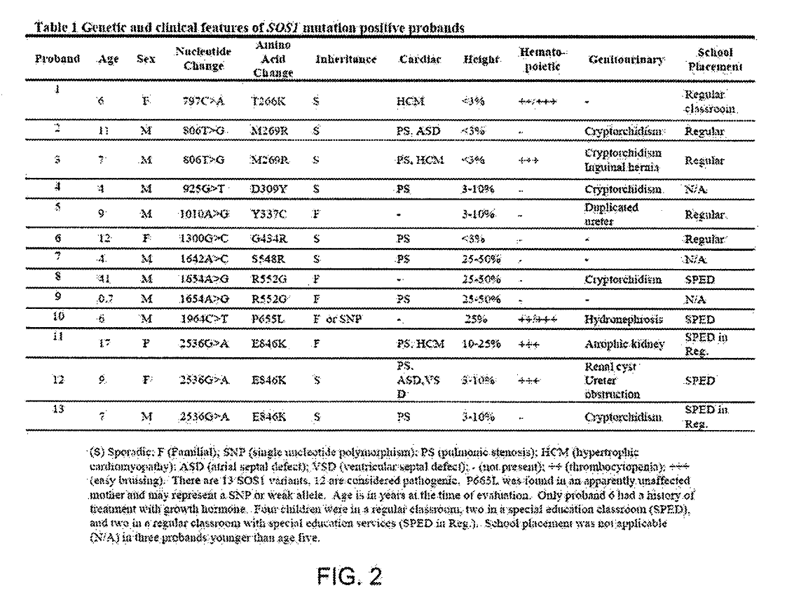

Fourteen patients with SOS1 variants were identified in our cohort of PTPN11-negative NS cases (FIG. 1a-d). One proband (and an unaffected parent) had a previously reported, although not validated, SNP (encoding N1011S) in SOS1 (see the World Wide Web at www.ncbi.nlm.nih.gov/SNP). Thirteen had one of nine novel missense changes, affecting six exons: T266K, D309Y, Y337C, G434R, S548R, and P655L (each found in a single proband); M269R (two probands); R552G (two probands), and E846K (three probands) (FIG. 2, Table 1). None of the identified variants were found in 188 chromosomes from normal individuals or in the public SNP database (on the World Wide Web at www.ensembl.org). Among the SOS1 mutation-positive cases, four were known to be familial. In three, the affected parent was found to have the same mutation, as expected; the parents of the fourth are deceased. Nine cases were judged to be sporadic. In five, analysis of the parents confirmed that these are indeed de novo mutations. In three cases, we were unable to obtain parental samples. However, in one sporadic case an apparently unaffected mother was found to have the same SOS1 allele (P655L) as their affected child. We cannot be sure if P655L or N1011S are bona fide mutations or rare polymorphisms, so we conservatively place the prevalence of SOS1 mutations in our PTPN11-negative NS cohort at ˜20% (12/59).

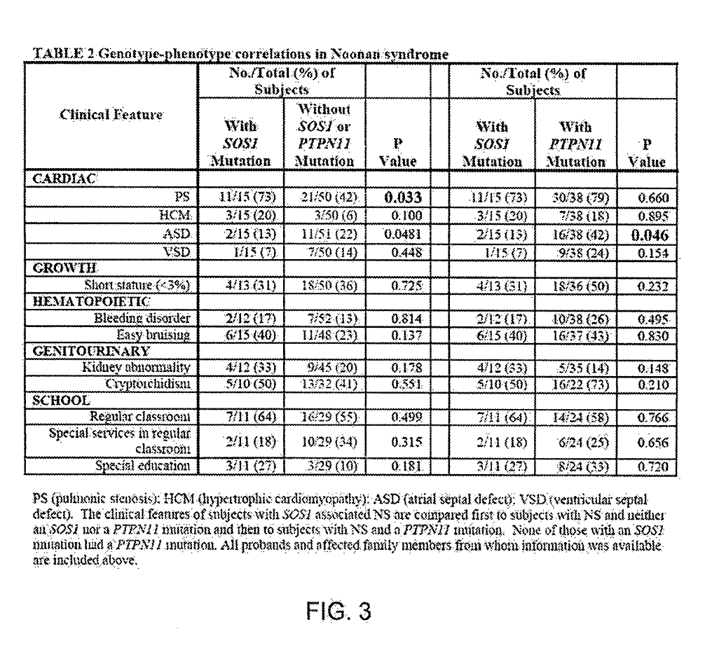

We asked whether the phenotypes differed in NS patients with PTPN11 mutations, SOS1 mutations or neither. NS patients in all three groups exhibited the typical facies characteristic of the syndrome (data not shown), and also shared most other phenotypic features (FIG. 3, Table 2). Our analysis did reveal two significant differences, however: pulmonic stenosis (PS) was more frequent in NS caused by SOS1 mutations than in those without SOS1 or PTPN11 mutations, whereas atrial septal defect (ASD) was more common in patients with PTPN11 mutations than in those with SOS1 mutations (FIG. 3, Table 2). Analysis of larger cohorts will be important to determine if there are additional genotype-phenotype correlations.

The NS-associated mutations map to multiple sub-domains within the SOS1 protein. SOS1 contains a RAS-GEF (Cdc25) domain, as well as conserved Histone-like Fold (HF), Dbl Homology (DH) and Pleckstrin Homology (PH) domains, a Helical Linker (HL), a RAS Exchange Motif (REM), and a proline-rich region (FIG. 1a). Although it binds the HL in vitro the function of the HF domain is unclear (Sondermann et al., Structure 11:1583-1593, 2003; Sondermann et al., Proc. Natl. Acad. Sci. 102:16632-16637, 2005). The DH domain may act as a GEF for Rac family small G proteins (Rac-GEF), whereas the PH domain is thought to regulate this GEF activity by permitting access to Rac only upon phosphoinositide binding (Nimnual and Bar-Sagi, D, Science STKE 145:1-3, 2002; Soisson et al., Cell 95(2):259-68, 1998). Structural studies revealed that SOS1 has two RAS binding sites. In addition to the “effector” site in the Cdc25 domain, the REM and Cdc25 domains form a second (“allosteric”) RAS binding site. When bound at the allosteric site, RAS enhances the RAS-GEF activity of the Cdc25 domain in vitro (Margarit et al., Cell 112:685-695, 2003; Sondermann et al., Cell 119:393-405, 2004). The DH domain competes with RAS for binding to the allosteric site, which led Sonderman et al. to suggest that the DH domain also acts as an intramolecular inhibitor of RAS-GEF activity (Sondermann et al., Cell 119:393-405, 2004).

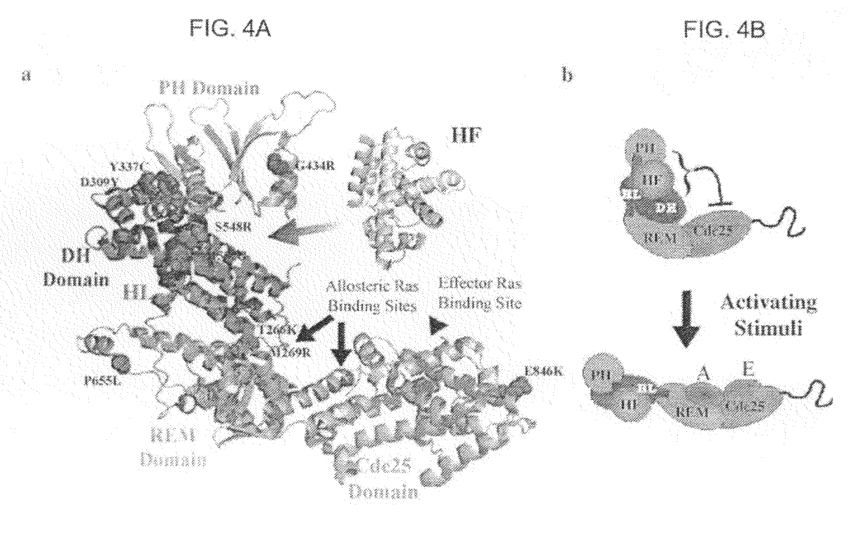

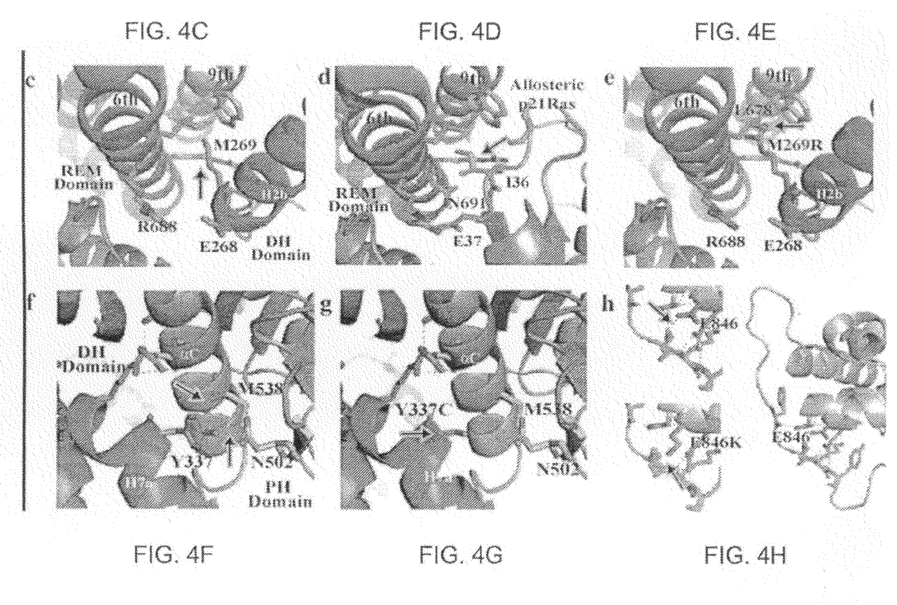

To obtain clues to their possible consequences, the SOS1 mutant residues were positioned on available crystal structures (containing the DH/PH, HL, REM and Cdc25 domains, and the REM and Cdc25 domains with bound RAS, respectively) (Margarit et al., Cell 112:685-695, 2003; Sondermann et al., Cell 119:393-405, 2004). Five (T266K, M269R, D309Y, Y337C, and G434R) lie within the DH/PH module (FIGS. 4a). T266 and M269 lie at the interface between the DH and REM domains, with M269 occupying a hydrophobic pocket in the REM domain that binds to allosteric RAS (FIG. 4c,d), and T266 required to position M269 properly (data not shown). Mutation of either of these residues (FIG. 4d and data not shown) should disrupt the DH/REM domain interface, allowing increased access of RAS to the allosteric site and potentially enhancing RAS-GEF activity. Indeed, an E268A/M269A/D271A SOS1 triple mutant signficantly enhances RAS-GEF activity in vitro, although the physiological effects of mutation have not been tested (Margarit et al., Cell 112:685-695, 2003). Y337 and D309 are located within the DH domain near the DH/PH interface. Y337C should result in loss of contact with several residues in the PH domain, probably reducing DH/PH domain interaction (FIG. 44f,g), D309 is solvent-exposed (FIG. 4a), but its replacement by a large, hydrophobic tyrosine (D309Y) also might cause a conformational change affecting the DH/PH binding interface. Although disruption of DH/PH domain interactions might enhance RAC activation, it is not immediately clear from the structure how (or if) this would affect RAS activation (but see below). R552 and S548 are adjacent residues in the HL. Alanyl substitution for R552 ablates binding between the HF and HL (Sondermann et al., Proc. Natl. Acad. Sci. 102:16632-16637, 2005); presumably, R5520 and S548R also would disrupt HF/HL binding. G434 is solvent-exposed, but lies close to the predicted HF binding site, so the G434R mutation might also disrupt HF/FL interaction. The Cde25 domain residue E846 forms a salt bridge with K1029, which lies within an extended loop across the end of the Cdc25 domain. E846K mutation would disrupt this salt bridge, probably causing displacement of this loop (FIG. 4h). However, the function of this loop and the physiological consequences of its displacement are not known.

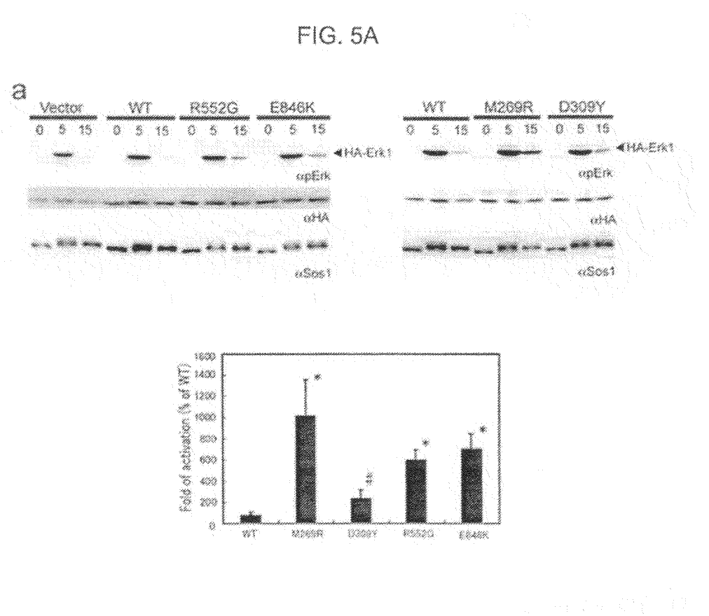

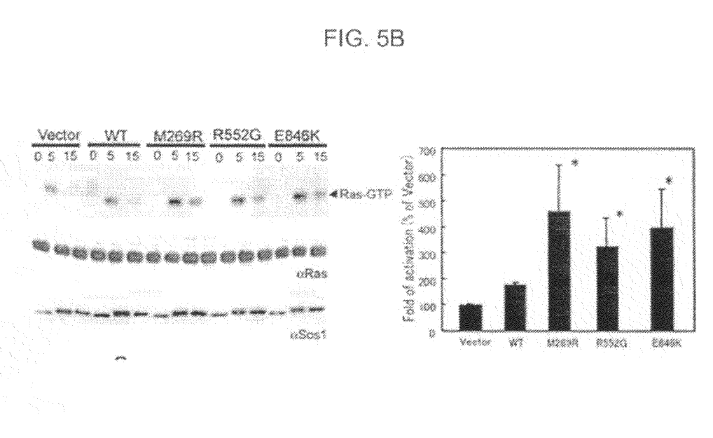

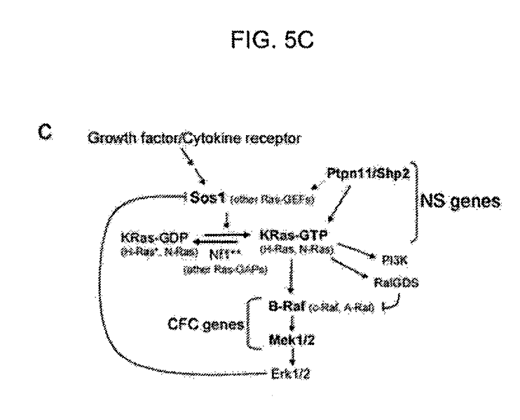

The above analysis (together with the genetics of NS) suggested that NS-associated SOS1 mutations are hypermorphs. To directly assess their activity in vivo, the effects of WT SOS1 and five representative mutants (M269R, D309Y, R552G, E846K and Y337C) on EGF-stimulated RAS/ERK activation were compared. Under the conditions used, each SOS1 protein was expressed at ˜3-4-fold higher levels than endogenous SOS1 (FIG. 5, legend). WT SOS1 slightly enhanced ERK activation (as monitored using a co-transfected HA-ERK construct) at five minutes post-EGF addition, but had no effect on ERK activity at later times. However, four of the mutants (M269R, D309Y, R552G and E846K) caused EGF-evoked ERK activation to be sustained significantly, with M269R having the greatest effect (FIG. 5a). These mutants also caused sustained activation of endogenous RAS, with M269R significantly enhancing RAS activation even at 5 minutes post-stimulation (FIG. 5b and data not shown). Notably, the effects of these NS-associated SOS1 mutants were strikingly similar to those of NS-associated PTPN11 mutants assayed under similar conditions (Fragale et al., Hum. Mut. 23:267-277, 2004; Kontaridis et al., J. Biol. Chem. 281: 6785-6792, 2005). No enhancement of ERK activation was seen in response to transfection of Y337C expression constructs; however, the Y337C protein was unstable and did not accumulate significantly in transfected cells. Our results identify gain-of-function SOS1 mutations as a major cause of NS. Although overall, the phenotype of NS patients is remarkably similar, a difference was detected in the prevalence of cardiac abnormalities (PS, ASD) that depends on the causal gene (PTPN11, SOS1 or unknown). Interestingly, although ASD appears to be relatively rare in SOS1 mutation-positive (compared to PTPN11 mutation-positive) NS, of the two SOS1 probands with ASD, one had the most activating mutation (M269R) and the other had the second most activating mutation (E846K) among those tested (FIG. 5). Knock-in mice expressing the highly activated NS-associated PTPN11 mutant, D61G, have a much higher penetrance of ASD than those expressing the weaker N308D allele (data not shown) (Keilhack et al., J Biol Chem 280:30984-30993, 2005; Araki et al., Nat. Med. 10:849-857, 2004). Together, these data suggest that the degree of activation of SOS1 or SHP2, and probably, the level of ERK hyperactivation, is a key determinant of whether septal defects occur in NS. Nevertheless, the striking phenotypic similarity across our NS cohort further emphasizes the intimate involvement of SHP2 and SOS1 in RAS activation, and strongly suggests that the remaining, as yet undiscovered, NS genes will encode proteins that act at or upstream of the level of RAS (e,g., other RAS-GEFs/RAS family members).

Our findings also provide further insights into the differences between NS and CFC. Although both likely are caused enhanced ERK activation, CFC mutations affect downstream components of the RAS/ERK pathway (BRAF, MEK1/2), whereas more proximal components (PTPN11/SOS1/KRAS) are mutated in NS. In addition to increasing ERK activation, NS (in contrast to CPC) mutants could affect other downstream pathways; e.g., RAS proteins have additional effectors besides RAF, and SOS1 can act as both a RAC-GEF and RAS-GEF. Alternatively, differences in negative feedback pathways that act on proximal and distal components of the RAS/ERK cascade may account for phenotypic differences between NS and CFC (Bentires-Alj et al., Nat. Med. 12:11-13, 2006). Indeed, both of the above possibilities may contribute, as the RAS effector RAL-GDS can antagonize other RAS effector functions in some cellular contexts (Goi et al., Mol Cell. Biol. 19:1731-1741, 1999).

The novel SOS1 mutations associated with NS provide insights into SOS1 regulation. The hypermorphic effects of M269R and D309Y provide strong genetic and biochemical evidence that, as suggested previously, the DH/PH module inhibits RAS-GEF activity by controlling binding of allosteric RAS (Sondermann et al., Cell 119:393-405, 2004). Furthermore, the R552S mutant indicates that HF/HL interaction, previously defined only by in vitro studies, has a key role in auto-inhibition of RAS-GEF activity (Sondermann et al., Proc. Natl. Acad. Sci. 102:16632-16637, 2005). Together with these previous studies, the data herein suggest that the entire SOS1 N-terminus may function as a unit to inhibit the REM/Cdc25 domain, with mutations that disrupt any of these interactions, or even physiological stimuli that affect these interactions (e.g., phosphoinositide binding to the PH domain (Soisson et al., Cell 95(2):259-68, 1998)), resulting in enhanced RAS-GEF activity. Finally, the activating effects of the E846K mutant (FIG. 4h, 5a,b) suggest that the extended loop in the Cdc25 domain containing K1029 has a previously unsuspected regulatory role.

To our knowledge, the SOS1 alleles associated with NS represent the first example of GEF mutations associated with human disease. Activating RAS mutations or homozygous deficiency of the RAS-GAP NF1 (the gene for Neurofibromatosis-Type 1) are associated with several human malignancies (Bos, Cancer Research 49:4682-4689, 1989; Cichowski et al., Science 286:2172-2176, 1999). NS patients are at increased risk of myeloproliferative disease and certain leukemias, particularly juvenile myelomonocytic leukemia (JMML), and somatic mutations of the other two NS genes. PTPN11 and KRAS, are found in ˜60% of sporadic JMML and in other neoplasms (Tartaglia et al., Annu Rev Genomics Hum Genet 6: 45-68, 2005; Lauchle et al., Pediatr Blood Cancer 46:579-585, 2005). Accordingly, the results here also implicate SOS1 as a potential human protooncogene in leukemias and solid tumors.

Methods

Subjects. DNA samples were obtained from individuals given a clinical diagnosis of NS by a medical geneticist. Each was either examined and had records reviewed by A.R. (75%), or had photos and records evaluated by A.R (25%). Inclusion criteria were based upon the van der Burght system (van der Burgt et al., Am. J. Med. Genet. 53:187-191, 1994): three or more facial features and one other major or two minor criteria, or two facial features and two major or three minor criteria. Facial features included broad forehead, hypertelorism, down-slanting palpebral fissures, highly arched palate, and low set, posteriorly rotated ears. Major criteria included PS, hypertrophic cardiomyopathy, typical electrocardiogram, height<3%, pectus carinatum and/or excavatum, first degree relative with NS, or all three of eryptorchidism, mental retardation, and lymphatic dysplasia. Minor criteria, included: other cardiac defect, height<10%, broad thorax, first degree relative with features suggestive of NS, or one of: cryptorchidism, mental retardation, or lymphatic dysplasia. Genotype-phenotype correlations were evaluated by using 2×2 contingency-table analysis. The significance threshold was set at P<0.05.

Mutation detection. Genomic DNA was extracted from whole blood or another tissue from each enrollee at the Harvard/Partners Laboratory for Molecular Medicine (CLIA#22D1005307). Each sample was subjected to bi-directional DNA sequencing of the 15 coding exons of PTPN11. The primer sequences are shown in Table C.

TABLE C
Primer pairs used to amplify the SOS1 coding sequence

Primer Sequence
(5′-3′)

| | | |
|---|---|---|
| Exon | Forward Sequence | Reverse Sequence |

| | | |
|---|---|---|
| 1 | CTGGTACCTGTGTCGGGTG | TCCAACTGTTGTTGTCCTGG |
| 2 | CCAGGGTGGTCTCAAACTC | TGTTCCCAAGCATTCTGGAT |
| 3 | TCAGGATGTGATATTCCCCC | AGCATCCCTTCTCACCACAT |
| 4 | TGTTGTTGGTAAGCACAGGC | CCCTAGTTGCAACAGCACAG |
| 5 | CAGAGAAAACCAAGTCAGGGA | TCATGCAAATTTCACAACACA |
| 6 | TGAAGGAGTCCTGAGACCAGA | TGACCCTATGAAAAGGAGCA |
| 7-8 | ATTGTGCTCGCATAGTCGTG | AAATTCACACTTGAATATGTTACAAA |
| 9 | TGCTCCCCATTTCTTTTCAG | CTTGAGGAGGGAACTGGGAT |
| 10 cut 1 | CATGAGCTCTAGGTTTTCTGTCA | CTAGGCAGCCTCATCTGCTC |
| 10 cut 2 | GATGACACCAATGAATACAAGCA | GCCAAGTGACCTCATTTTCTC |
| 11 | GCAGTGCATTACCAAGTCCA | TTGTTCACTGACAAGTTCCATTTT |
| 12 | TTTGCTGACTGGTGAAAACG | ACCCAGTGCCGACATACATT |
| 13 | AGGACCTTTCCCAAACAAGG | CAGGGTGACTTGAGCCATTT |
| 14 | AAGATTAGGATTGGGGACCG | AGAGGGACCAGGGGAGTAAA |
| 15 | GTTTTCACAGACCTTTCTGTTGG | AAAGAAAATTAAAATTAGGTGAGTGTG |
| 16 | GGGAAAGAGCAGTATGCCTG | CTTAGGCTGGGACCTGTGAA |
| 17 | ATTTGGGCGTTTCTGTTAGC | GAGTTTGGCTATGCCTCTGC |
| 18 | TGTTTTGGCAACTGAGATGG | GCTATTTCCCATCGGATTCA |
| 19 | TCCAGCAGTACCAAAATCAGC | GGAAGTGGGATATTCCTGGAC |
| 20 | GCTGAATTTTACCAGGCACA | GGCTGTTCCAGCAGATTTTT |
| 21 | GCCCCAAGAACATTTTTGAA | TGCCAGACCCAAGAAGAGTT |
| 22 | TCTGCATGCTTTTATGGCAG | GAACTGCTTGAAACCAAATTCC |
| 23 cut 1 | GCATGTTTGAAAACCCCAAC | GAACTCAGGAAGAATGGGCA |
| 23 cut 2 | GTGGACCTTCATTCCATTGC | AAAAACTGTCATGTGGGCCT |

Individuals who did not exhibit pathogenic mutations in PTPN11 were selected for high throughput analysis for mutations in other candidate genes, including BRAF, CSK, PTPN6, PAG, MRAS, SOS1 and SOS2. Primers were designed to amplify coding regions of all genes plus flanking intronic sequences. Each primer, 20-24 bases in length, was designed to have a calculated Tm of 60-62° C., and pairs were designed to generate 400-600 base pair products. M13 forward or reverse primer tags were appended to facilitate sequencing. All primers were screened against dbSNP to exclude known polymorphisms, and primers were validated for robust amplification of unique products using 3 control DNAs. The DNA used for analysis of BRAF, CSK, PTPN6, PAG and MRAS was genomic DNA from the PTPN11-negative individuals described above. For SOS1 and SOS2, whole genome amplification (WGA) was performed on aliquots of genomic DNA from the PTPN11-negative individuals, and the products used for analysis. All 23 coding exons of SOS1, including the variant Exon 22 and at least 30 intronic base pairs flanking each exon were sequenced. PCR products were purified using AMPure beads (Agencourt) and then sequenced using ABI BigDye 3.1 dye terminator chemistry and a 3730XL DNA analyzer. Data were transferred to the UNIX Platform and assembled and analyzed using PolyPhred v5.0 (Stephens, 2006) and Consed (Gordon, 1998). DNA sequence variants were independently reviewed and confirmed using Mutation Surveyor (Softgenetics). Mutations identified were subsequently confirmed by amplification and sequence analysis of aliquots of the original genomic DNA.

Biochemical analyses. 293T cells were maintained in DMEM plus 10% fetal bovine serum and antibiotics. A cDNA for human SOS1 isoform I was purchased from Origene. After correcting a point mutation found in the initial clone, NS-associated SOS1 variants were generated by PCR-directed mutagenesis, and cloned into the retroviral vector pBABE-puro. WT or NS-associated SOS1 expression constructs were transiently translated into 293T cells using polyethylenimine (Godbey et al., Gene Ther. 6:1380-1388, 1999). Where indicated, an HA-ERK expression construct was co-transfected. Twenty-four hours post-transfection, cells were starved in serum-free DMEM for 8-12 hours before stimulation with EGF (20 ng/ml). ERK activation was detected by immunoblotting with anti-pERK antibodies (Cell Signaling), followed by reprobing with anti-HA monoclonal antibody (12CA5) to control for loading, and anti-SOS1 antibodies (Santa Cruz) to assess SOS1 expression. RAS activation was assessed using GST-RAF-RBD beads, as described previously (Kontaridis et al., J. Biol. Chem. 281: 6785-6792, 2005.). Band intensities were quantified using NIH Image.

REFERENCE SEQUENCES (GenBank® Acc. Nos.): PTPN11, NM_{—}002834; CSK, NM_{—}004383: PTPN6, NM_{—}080549; PAG, NM_{—}018440; MRAS, NM_{—}012219: SOS1, NM_{—}005633; SOS2, NM_{—}006939; BRAF, NM_{—}004333.

### Example 2

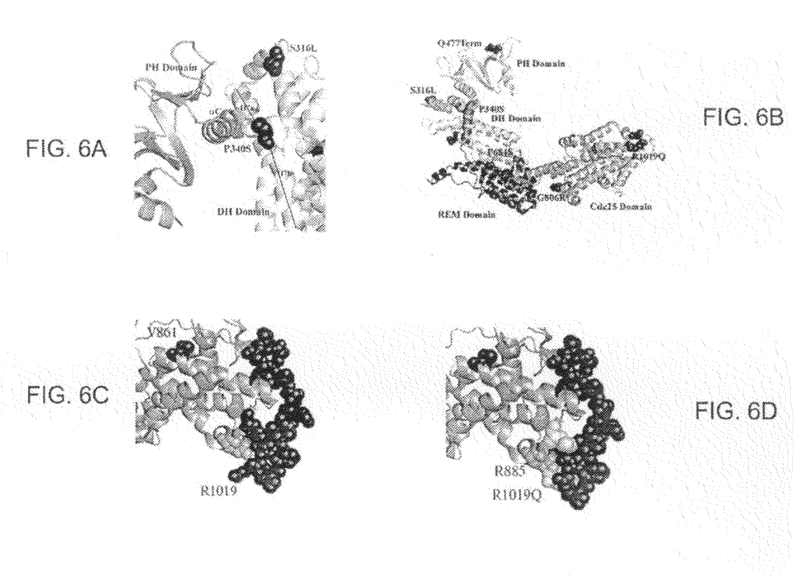

The SOS1 gene mutations listed in Table D were identified in a screen of 96 colon cancer samples. The following is a synopsis of structural analysis of various mutations. S316 sits within the Dbl Homology (DH) domain near the DH/PH domain interface at the end of an alpha helix. S316L may interfere with the termination of this helix or alter the shape of the loop extending from it (FIG. 6A). This may result in the disruption of the interaction between the DH and the PH domains that is thought to be inhibitory to Rac and possibly Ras exchange activity.

The interaction between the DH and PH domains take place between the αC helix of the PH domain and the Helix H7a of the DR domain. P340 lies in H7b, which is actually contiguous with H7a. These ‘two’ helixes are separated by the kink induced by P340. H7b is bounded by the other structural helixes in the DH domain and the deflection (FIG. 6A, black line on image) of the helix caused by P340 aligns residues necessary for creation of the binding interface between the DH and PH domains. P340S would result in the straightening of the helix and the subsequent displacement of H7a away from the αC helix reducing the ability of these two domains to bind each other.

Q477 to Stop results in a truncated protein consisting of the histone fold, the DH domain and a third of the PH domain (FIG. 6B). The histone fold (not shown) is thought to aid in membrane targeting. The DH domain's Rac-GEF activity is thought to be inhibited by the PH domain in the absence of phosphoinositide, suggesting that the Q477 truncation mutant produces an unregulated Rac-GEF protein.

The interaction between the REM and DH domains appears critical for the regulation of Sos-1 Ras-GEF activity. P684 lies in a helix within the REM domain that lies adjacent to the DH domain. The shape of the interface between the two domains requires the P684 containing helix to deflect away from the DH domain at the position of P684 (FIG. 6B). P684 would result in a straightening of this helix resulting in collision between the REM and DH domain precluding their interaction. This should cause dysregulation of the Cde25 domain's catalytic activity.

G806 is located near the interface between the REM and Cdc25 domain and G806R may alter the interactions between these two domains, which may lead to the activation of the Cdc25 domain's Ras-GEF activity through a decrease in auto-inhibition (FIG. 6B).

V861 lies buried within the Cdc25 domain and may cause an alteration in the rate of Ras binding and/or nucleotide release (FIGS. 6C and 6D)

R1019 lies at the beginning of an extended loop that lies across the distal end of the Cdc25 domain. R1019Q is not accommodated within the structure, possibly leading to a displacement of the loop from its wild-type position.

TABLE D
Cancer-associated SOS1 Gene Mutations

| | | | | | Results of |
|---|---|---|---|---|---|
| Sample | Amino Acid | | Nucleotide | | Confirmation |
| ID | Change | Exon | Change | | Sequencing |
| MX15 | SOS1−116G > T | 5′UTR?? | none | SNP | |
| MX16 | SOS1:R65 | 2 | CGA > CGC | synonymous | |
| HX108 | SOS1:S316L | 7 | TCA > TTA | missense | Confirmed in |
| | | | | | tumor whole |
| | | | | | genome |
| | | | | | amplification |
| | | | | | (WGA), neg. in |
| | | | | | normal |
| HX130 | SOS1:P340S | 8 | CCC > TCC | missense | Confirmed in |
| | | | | | tumor WGA, neg. |
| | | | | | in normal |
| CO85 | SOS1: | IVS8 | SNP | Possible | Seen in Reverse |
| | IVS8 + 5G > C | | | Splice Variant | only, tumor WGA |
| | | | | | and normal |
| MX15 | SOS1: | IVS8 | SNP | Possible | Seen in Reverse |
| | IVS8 + 5G > C | | | Splice Variant | only, tumor WGA |
| | | | | | and normal |
| MX30 | SOS1: | IVS8 | SNP | Possible | Seen in Reverse |
| | IVS8 + 5G > C | | | Splice Variant | only, tumor WGA |
| | | | | | and normal |
| HX133 | SOS1:G414 | 10 | GGG > GGA | synonymous | |
| HX136 | SOS1:Q477X | 10 | CAG > TAG | termination | Confirmed in |
| | | | | | tumor WGA, also |
| | | | | | in normal |
| HX107 | SOS1:P655L | 12 | CCA > CTA | missense | Confirmed in |
| | | | | | tumor WGA, also |
| | | | | | in normal |
| HX125 | SOS1:P684S | 12 | CCT > TCT | missense | Confirmed in non- |
| | | | | | WGA, neg. in |
| | | | | | normal |
| HX110 | SOS1:G806R | 15 | GGA > AGA | missense | Confirmed in |
| | | | | | WGA, non-WGA |
| | | | | | neg. in normal |
| HX164 | SOS1:V861I | 16 | GTC > ATC | missense | Confirmed in |
| | | | | | WGA, neg. in |
| | | | | | normal (ss) |
| HX63 | SOS1:R1019Q | 19 | CGA > CAA | missense | Confirmed in |
| | | | | | WGA, neg. in |
| | | | | | normal |
| HX125 | SOS1:P1236 | 23 | CCC > CCT | synonymous | |

A number of embodiments of the invention have been described. Nevertheless, it will be understood that various modifications may be made without departing from the spirit and scope of the invention. Accordingly, other embodiments are within the scope of the following claims.

1. A method for diagnosing in a subject, or identifying a subject at risk for, Noonan syndrome (NS), the method comprising:
determining if one or more mutations are present in a Son of Sevenless 1 (SOS1) gene or SOS1 polypeptide of the subject, wherein the presence of one or more mutations indicates that the subject is affected with, or at risk for, NS.

2.-9. (canceled)

10. The method of claim 1, wherein the method further comprises determining whether any one or both of a PTPN11 gene and a KRAS gene of the subject comprises a mutation.

11.-14. (canceled)

15. The method of claim 1, wherein the mutation comprises a mutation at one of the following nucleotide positions of the SOS1 sequence of SEQ ID NO:1: 797, 806, 925, 1010, 1358, 1642, 1654, 1964, and 2536.

16. (canceled)

17. The method of claim 1, wherein the mutation is one of the following substitutions: 797C>A, 806T>G, 925G>T, 1010A>G, 1358G>C, 1642A>C, 1654A>G, 1964C>T, or 2536G>A.

18.-19. (canceled)

20. The method of claim 1, wherein the mutation results in a mutation at one of the following amino acid positions in the SOS1 polypeptide of SEQ ID NO:2: T266, M269, D309, Y337, G434, S548, R552, P655, or E846.

21. (canceled)

22. The method of claim 20, wherein the mutation is one of the following substitutions: T266K, M269R, D309Y, Y337C, G434R, S548R, R552G, P655L, or E846K.

23. The method of claim 1, wherein the mutation in the SOS1 gene results in a mutation in one of the following domains of the polypeptide encoded by the gene: the Dbl Homology (DH) domain, the Pleckstrin Homology (PH) domain, the Helical Linker (HL) domain, the Ras Exchange Motif (REM) domain, or the Cdc25 domain.

24.-45. (canceled)

46. The method of claim 1, further comprising evaluating whether the subject
is at risk for developing, or is affected by, pulmonary stenosis or an atrial septal defect.

47.-49. (canceled)

50. The method of claim 1, wherein the
subject has one or more characteristics or symptoms of NS, cardio-facial-cutaneous syndrome, or Costello syndrome.

51.-87. (canceled)

88. The method of claim 1, wherein determining if one or more mutations are present in the SOS1 polypeptide of the subject comprises evaluating the expression or activity of a SOS1 polypeptide in a sample from the subject, relative to a control, and wherein an increase in the expression or activity of the SOS1 polypeptide relative to the control is indicative of Noonan syndrome.

89. The method of claim 88, wherein enhanced Ras and/or Erk activation mediated by the SOS1 polypeptide, relative to a control, is indicative of Noonan syndrome.

90. A method for identifying an agent that modulates the activity of a SOS1 polypeptide, the method comprising:
providing a sample comprising a SOS1 polypeptide,
contacting the sample with a test compound under conditions in which the SOS1 polypeptide is active, and
evaluating the activity of the SOS1 polypeptide in the presence of the test compound, wherein a change in the activity of the SOS1 polypeptide indicates that the test compound is an agent that modulates the activity of the SOS1 polypeptide.

91. The method of claim 90, wherein the SOS1 polypeptide is a mutant polypeptide.

92. The method of claim 90, further comprising evaluating the compound for an effect on cell growth.

93. (canceled)

94. The method of claim 90, further comprising evaluating an effect of the compound on a symptom of Noonan Syndrome.

95. A method for genotyping a subject, the method comprising:
determining the identity of at least one nucleotide of a SOS1 gene of a subject, and
creating a record which includes information about the identity of the nucleotide and information relating to a genotypic or phenotypic characteristic of Noonan syndrome or a neoplastic disorder in the subject.

96. The method of claim 95, wherein the method further includes comparing the information in the record to reference information.

97. The method of claim 95, wherein the method further includes comparing the nucleotide to a corresponding nucleotide from a genetic relative or family member.

98. The method of claim 95, wherein the method further includes evaluating risk or determining diagnosis of Noonan syndrome or a neoplastic disorder in the subject as a function of the information in the record.

99.-128. (canceled)
